SEQUENCE LISTING (218 sequences)

SEQ ID NO: 1 - protein, 17 aa, unidentified
YGYGGLYGGLYGGLGYG

SEQ ID NO: 2 - protein, 17 aa, unidentified
YGYGGLFGGLFGGLGYG

SEQ ID NO: 3 - protein, 17 aa, unidentified
YGFGGLYGGLFGGLGFG

SEQ ID NO: 4 - protein, 7 aa, unidentified
PSTGTLS

SEQ ID NO: 5 - protein, 6 aa, unidentified
PSTGTL

SEQ ID NO: 6 - protein, 5 aa, unidentified
PSTGT

SEQ ID NO: 7 - protein, 4 aa, unidentified
PSTG

SEQ ID NO: 8 - protein, 6 aa, unidentified
STGTLS

SEQ ID NO: 9 - protein, 5 aa, synthetic construct
STGTL

SEQ ID NO: 10 - protein, 4 aa, unidentified
STGT

SEQ ID NO: 11 - protein, 11 aa, unidentified
AATAVHTTHHA

SEQ ID NO: 12 - protein, 13 aa, unidentified
VAHHSVVSRRYAI

SEQ ID NO: 13 - protein, 10 aa, unidentified
SATAVSHTSH

SEQ ID NO: 14 - protein, 12 aa, unidentified
VGAAVSHVTHHA

SEQ ID NO: 15 - protein, 11 aa, unidentified
HAVGAVSTLHH

SEQ ID NO: 16 - protein, 11 aa, unidentified
AAAVSHVTHHA

SEQ ID NO: 17 - protein, 12 aa, unidentified
VATVTSQTSHHV

SEQ ID NO: 18 - protein, 10 aa, unidentified
AASAVSTSTH

SEQ ID NO: 19 - protein, 11 aa, unidentified
ASSAVSHTSHH

SEQ ID NO: 20 - protein, 9 aa, unidentified
HSVAVGVHH

SEQ ID NO: 21 - protein, 9 aa, unidentified
HTVSHVSHG

SEQ ID NO: 22 - protein, 9 aa, unidentified
VTSAVHTVS

SEQ ID NO: 23 - protein, 14 aa, unidentified
VGQSVSTVSHGVHA

SEQ ID NO: 24 - protein, 13 aa, unidentified
VAHHGTISRRYAI

SEQ ID NO: 25 - protein, 15 aa, unidentified
TGASVNTVSHGISHA

SEQ ID NO: 26 - protein, 14 aa, unidentified
VGASVSTVSHGIGH

SEQ ID NO: 27 - protein, 14 aa, unidentified
VGSTISHTTHGVHH

SEQ ID NO: 28 - protein, 14 aa, unidentified
AATSNSHTTHGVHH

SEQ ID NO: 29 - protein, 15 aa, unidentified
YYRKSVSTVSHGAHY

SEQ ID NO: 30 - protein, 13 aa, unidentified
HVGTSVHSVSHGA

SEQ ID NO: 31 - protein, 11 aa, unidentified
ATAVSHTTHHA

SEQ ID NO: 32 - protein, 14 aa, unidentified
VSSSVSHVSHGAHY

SEQ ID NO: 33 - protein, 13 aa, unidentified
VSSVRTVSHGLHH

SEQ ID NO: 34 - protein, 10 aa, unidentified
RSVSHTTHSA

SEQ ID NO: 35 - protein, 15 aa, unidentified
AVSTVSHGLGYGLHH

SEQ ID NO: 36 - protein, 15 aa, unidentified
YIGRSVSTVSHGSHY

SEQ ID NO: 37 - protein, 11 aa, unidentified
AVGHTTVTHAV

SEQ ID NO: 38 - protein, 11 aa, unidentified
AATTYRQTTHH

SEQ ID NO: 39 - protein, 15 aa, unidentified
YYRRSFSTVSHGAHY

SEQ ID NO: 40 - protein, 13 aa, unidentified
AATSVKTVSHGFH

SEQ ID NO: 41 - protein, 11 aa, unidentified
AATAVSPHNSS

SEQ ID NO: 42 - protein, 14 aa, unidentified
AATAVSHTTHGIHH

SEQ ID NO: 43 - protein, 9 aa, unidentified
AATTAVTHH

SEQ ID NO: 44 - protein, 13 aa, unidentified
HVGTSVHSVSHGV

SEQ ID NO: 45 - protein, 12 aa, unidentified
TGSSISTVSHGV

SEQ ID NO: 46 - protein, 9 aa, unidentified
VVSHVTHTI

SEQ ID NO: 47 - protein, 14 aa, unidentified
AASSVTHTTHGVAH

SEQ ID NO: 48 - protein, 13 aa, unidentified
VTHYSHVSHDVHQ

SEQ ID NO: 49 - protein, 11 aa, unidentified
AATTAVTQTHH

SEQ ID NO: 50 - protein, 14 aa, unidentified
MSSSVSHVSHTAHS

SEQ ID NO: 51 - protein, 12 aa, unidentified
ASTSVSHTTHSV

SEQ ID NO: 52 - protein, 12 aa, unidentified
TSVSQVSHTAHS

SEQ ID NO: 53 - protein, 10 aa, unidentified
GHAVTHTVHH

SEQ ID NO: 54 - protein, 14 aa, unidentified
AATTVSHTTHGAHH

SEQ ID NO: 55 - protein, 17 aa, unidentified
SSYYGRSASTVSHGTHY

SEQ ID NO: 56 - protein, 13 aa, unidentified
VSSVSTVSHGLHH

SEQ ID NO: 57 - protein, 13 aa, unidentified
HIGTSVSSVSHGA

SEQ ID NO: 58 - protein, 9 aa, unidentified
HSVSHVSHG

SEQ ID NO: 59 - protein, 6 aa, unidentified
GAAFHY

SEQ ID NO: 60 - protein, 11 aa, unidentified
GVAAYSHSVHH

SEQ ID NO: 61 - protein, 14 aa, unidentified
VGASVSTVSHGVHA

SEQ ID NO: 62 - protein, 13 aa, unidentified
AATSVKTVSHGYH

SEQ ID NO: 63 - protein, 14 aa, unidentified
ATASVSHTTHGVHH

SEQ ID NO: 64 - protein, 11 aa, unidentified
HAVSTVAHGIH

SEQ ID NO: 65 - protein, 9 aa, unidentified
AVSHVTHTI

SEQ ID NO: 66 - protein, 10 aa, unidentified
VRYHGYSIGH

SEQ ID NO: 67 - protein, 11 aa, unidentified
AVRHTTVTHAV

SEQ ID NO: 68 - protein, 12 aa, unidentified
GATTYSHTTHAV

SEQ ID NO: 69 - protein, 10 aa, unidentified
VGGAVSTVHH

SEQ ID NO: 70 - protein, 12 aa, unidentified
AATTVSHSTHAV

SEQ ID NO: 71 - protein, 11 aa, unidentified
HASTTTHSIGL

SEQ ID NO: 72 - protein, 12 aa, unidentified
AVSHVTHTIPHA

SEQ ID NO: 73 - protein, 11 aa, unidentified
AAAVSHTTHHA

SEQ ID NO: 74 - protein, 14 aa, unidentified
TGSSISTVSHGVHS

SEQ ID NO: 75 - protein, 14 aa, unidentified
VASSVSHTTHGVHH

SEQ ID NO: 76 - protein, 14 aa, unidentified
SAGGTTVSHSTHGV

SEQ ID NO: 77 - protein, 9 aa, unidentified
SVATRRVVY

SEQ ID NO: 78 - protein, 14 aa, unidentified
AGSSISTVSHGVHA

SEQ ID NO: 79 - protein, 12 aa, unidentified
AATSVSHTTHSV

SEQ ID NO: 80 - protein, 10 aa, unidentified
HSVSTVSHGA

SEQ ID NO: 81 - protein, 12 aa, unidentified
TGTSVSTVSHGV

SEQ ID NO: 82 - protein, 15 aa, unidentified
VIHGGATLSTVSHGV

SEQ ID NO: 83 - protein, 14 aa, unidentified
SHGVSHTAGYSSHY

SEQ ID NO: 84 - protein, 15 aa, unidentified
VGSTSVSHTTHGVHH

SEQ ID NO: 85 - protein, 11 aa, unidentified
AATSYSHALHH

SEQ ID NO: 86 - protein, 12 aa, unidentified
AATTYSHTAHHA

SEQ ID NO: 87 - protein, 11 aa, unidentified
AATYSHTTHHA

SEQ ID NO: 88 - protein, 17 aa, unidentified
GLLGAAATTYKHTTHHA

SEQ ID NO: 89 - protein, 12 aa, unidentified
TGASVSTVSHGL

SEQ ID NO: 90 - protein, 33 aa, unidentified
GYGLGGLYGGYGLGGLHYGGYGLGGLHYGGYGL

SEQ ID NO: 91 - protein, 33 aa, unidentified
HYGVGGLYGGYGLGGLHGGYGLGGIYGGYGAHY

SEQ ID NO: 92 - protein, 27 aa, unidentified
GVGGYGMGGLYGGYGLGGVYGGYGLGG

SEQ ID NO: 93 - protein, 8 aa, unidentified
GYGLGVGL

SEQ ID NO: 94 - protein, 28 aa, unidentified
LGLGYGGYGLGLGYGLGHGYGLGLGAGI

SEQ ID NO: 95 - protein, 15 aa, unidentified
GLGLGYGYGLGHGLG

SEQ ID NO: 96 - protein, 12 aa, unidentified
GLGLGYGLGLGL

SEQ ID NO: 97 - protein, 30 aa, unidentified
MGGLYGGYGLGGVYGGYGLGGIYGGYGAHY

SEQ ID NO: 98 - protein, 33 aa, unidentified
GVGGLYGGYGLGGLYGGYGLGGLHGGYSLGGLY

SEQ ID NO: 99 - protein, 40 aa, unidentified
GGYGAHYGVGGLYGGYGLGGLHYGGYGLGGLHYGGYGLHY

SEQ ID NO: 100 - protein, 15 aa, unidentified
YGYGGLYGGLYGGLG

SEQ ID NO: 101 - protein, 33 aa, unidentified
VAYGGWGYGLGGLHGGWGYGLGGLHGGWGYALG

SEQ ID NO: 102 - protein, 22 aa, unidentified
GLYGGLHYVGLGYGGLYGGLHY

SEQ ID NO: 103 - protein, 19 aa, unidentified
VGYGGFGLGFGGLYGGLHY

SEQ ID NO: 104 - protein, 21 aa, unidentified
SLGAYGGYGLGGLIGGHSVYH

SEQ ID NO: 105 - protein, 21 aa, unidentified
SLGAYGGYGLGGIVGGYGAYN

SEQ ID NO: 106 - protein, 21 aa, unidentified
VGLGYGGFGLGYGGLYGGFGY

SEQ ID NO: 107 - protein, 12 aa, unidentified
VAYGGLGYGFGF

SEQ ID NO: 108 - protein, 13 aa, unidentified
GYGGLYGGLGYHY

SEQ ID NO: 109 - protein, 16 aa, unidentified
YGYGGLYGGLYGGLGY

SEQ ID NO: 110 - protein, 22 aa, unidentified
VGYGGYGLGAYGAYGLGYGLHY

SEQ ID NO: 111 - protein, 9 aa, unidentified
VGYAGYGLG

SEQ ID NO: 112 - protein, 9 aa, unidentified
YGGFGYGLY

SEQ ID NO: 113 - protein, 20 aa, unidentified
GYGGLYGHYGGYGLGGAYGH

SEQ ID NO: 114 - protein, 33 aa, unidentified
GIGGVYGHGIGGLGGVYGHGIGGVYGHGIGGLY

SEQ ID NO: 115 - protein, 51 aa, unidentified
GHGFGGAYGGYGGYGIGGVTYGGLGLGGLGYGGLGYGGLGYGGLGYGGLGY

SEQ ID NO: 116 - protein, 41 aa, unidentified
GGLGYGGLGYGGLGAGGLYGGAVGLGYGLGGGYGGLYGLHL

SEQ ID NO: 117 - protein, 12 aa, unidentified
ALGLGLYGGAHL

SEQ ID NO: 118 - protein, 12 aa, unidentified
GLGLNYGVYGLH

SEQ ID NO: 119 - protein, 20 aa, unidentified
GYGGWGYGLGGWGHGLGGLG

SEQ ID NO: 120 - protein, 17 aa, unidentified
YGGIGLGGLYGGYGAHF

SEQ ID NO: 121 - protein, 21 aa, unidentified
HSVGWGLGGWGGYGLGYGVHA

SEQ ID NO: 122 - protein, 21 aa, unidentified
ALGAYGGYGFGGIVGGHSVYH

SEQ ID NO: 123 - protein, 16 aa, unidentified
ALGGYGGYGLGGIVGG

SEQ ID NO: 124 - protein, 21 aa, unidentified
ALGAYGGYGLGGLVGGFGAYH

SEQ ID NO: 125 - protein, 33 aa, unidentified
VGFGGYGLGGYGLGGYGLGGYGLGGYGLGGLVG

SEQ ID NO: 126 - protein, 31 aa, unidentified
GYGSYHVGYGGYGLGGYGGYGLGGLTGGYGV

SEQ ID NO: 127 - protein, 15 aa, unidentified
GYGLGLGYGLGLGAG

SEQ ID NO: 128 - protein, 21 aa, unidentified
LGLGYGYGLGLGYGLGLGAGI

SEQ ID NO: 129 - protein, 17 aa, unidentified
HLGLGLGYGYGLGHGLG

SEQ ID NO: 130 - protein, 16 aa, unidentified
GLGLGYGLGLGYGYGV

SEQ ID NO: 131 - protein, 15 aa, unidentified
GYGLGLGLGGAGYGY

SEQ ID NO: 132 - protein, 19 aa, unidentified
VGGYGGFGLGGYGGYGLGG

SEQ ID NO: 133 - protein, 33 aa, unidentified
VGYGGLYGHYGGYGLGGVYGHGVGLGGVYGHGI

SEQ ID NO: 134 - protein, 44 aa, unidentified
GGAYGGYGLGVGGLYGGYGGYGIGGVGGYGGFGLGGYGGYGLGG

SEQ ID NO: 135 - protein, 33 aa, unidentified
VGYGGLYGHYGGYGLGGVYGHGVGLGGVYGHGV

SEQ ID NO: 136 - protein, 35 aa, unidentified
GLGGVYSHGIGGAYGGYGLGVGGLYGGYGGYGIGG

SEQ ID NO: 137 - protein, 19 aa, unidentified
VLSGGLGLSGLSGGYGTYR

SEQ ID NO: 138 - protein, 27 aa, unidentified
GYGGVGYGGLGYGGLGYGVGGLYGLQY

SEQ ID NO: 139 - protein, 26 aa, unidentified
GYGGWGYGLGGWGHGLGGLGSYGLHY

SEQ ID NO: 140 - protein, 21 aa, unidentified
HSVGWGLGGWGGYGLGYGVRS

SEQ ID NO: 141 - protein, 19 aa, unidentified
YGDVYGGLYGGLYGGLLGA

SEQ ID NO: 142 - protein, 33 aa, unidentified
VAYGGLGLGALGYGGLGYGGLGYGGLGAGGLYG

SEQ ID NO: 143 - protein, 16 aa, unidentified
LHYGYGLGLGLYGAHL

SEQ ID NO: 144 - protein, 26 aa, unidentified
AYGGWGYSLGRWGQGLGGLGTYGLHY

SEQ ID NO: 145 - protein, 21 aa, unidentified
ALGGYGGYGLGGIVGGHSVYH

SEQ ID NO: 146 - protein, 17 aa, unidentified
ALGEYGGYGLGGIVGGH

SEQ ID NO: 147 - protein, 21 aa, unidentified
GFGGYGLGGYGLGGYGLGGYG

SEQ ID NO: 148 - protein, 33 aa, unidentified
IGFGGWGHGYGYSGLGFGGWGHGLGGWGHGYGY

SEQ ID NO: 149 - protein, 20 aa, unidentified
HAVGFGGWGHGIGLGHGFGY

SEQ ID NO: 150 - protein, 14 aa, unidentified
HAVGFGGWGHGFGY

SEQ ID NO: 151 - protein, 19 aa, unidentified
HSVSYGGWGFGHGGLYGLH

SEQ ID NO: 152 - protein, 16 aa, unidentified
HADYGVSGLGGYVSSY

SEQ ID NO: 153 - protein, 33 aa, unidentified
VGFGGYGLGGYGLGGYGLGGYGLGGYGLGGVVG

SEQ ID NO: 154 - protein, 34 aa, unidentified
GFGGYHFGYGGVGYGGLGYGGLGYGVGGLYGLQY

SEQ ID NO: 155 - protein, 31 aa, unidentified
VAYGGLGLGALGYGGLGYGGLGAGGLYGLHY

SEQ ID NO: 156 - protein, 18 aa, unidentified
AGLGYGLGGVYGGYGLHA

SEQ ID NO: 157 - protein, 29 aa, unidentified
YGYGGLYGGLGYHAGYGLGGYGLGYGLHY

SEQ ID NO: 158 - protein, 14 aa, unidentified
VGWGLGGLYGGLHH

SEQ ID NO: 159 - protein, 18 aa, unidentified
GYGGYGLGLGGLYGGLHY

SEQ ID NO: 160 - protein, 18 aa, unidentified
GYGGYGLGFGGLYGGFGY

SEQ ID NO: 161 - protein, 24 aa, unidentified
AYGYGYGLGGYGGYGLYGGYGLHH

SEQ ID NO: 162 - protein, 29 aa, unidentified
VAYGGWGYGLGGLHGGWGYGLGGLYGGLH

SEQ ID NO: 163 - protein, 30 aa, unidentified
VGYAGYGYGLGSYGGYAGLGLGLYGAGYHY

SEQ ID NO: 164 - protein, 17 aa, unidentified
YAYGGLYGGYGLGAYGY

SEQ ID NO: 165 - protein, 30 aa, unidentified
VGYAGYGYGLGAYGGYAGLGLGLYGAGYHY

SEQ ID NO: 166 - protein, 15 aa, unidentified
VGYGGFGLAGYGYGY

SEQ ID NO: 167 - protein, 24 aa, unidentified
YGYGGLYGGYAGLGLGLYGAGYHY

SEQ ID NO: 168 - protein, 17 aa, unidentified
VGYAGYGLGLYGAGYHY

SEQ ID NO: 169 - protein, 33 aa, unidentified
VGYAGYGLGAYGGYAGYGLGAFGGYAGYGLGAF

SEQ ID NO: 170 - protein, 50 aa, unidentified
GGYAGLGLGLYGAGYHYLGFGGLLGGYGGLHHGVYGLGGYGGLYGGYGLG

SEQ ID NO: 171 - protein, 42 aa, unidentified
GYGLHGLHYLGFGGVLGYGGLHHGVYGLGGYGGLHGAYGLGG

SEQ ID NO: 172 - protein, 58 aa, unidentified
YGGLHGAYGLGGYGGLYGGYGLGGHVGYGGYGYGGLGAYGHYGGYGLGGLYGGYGLGG

SEQ ID NO: 173 - protein, 40 aa, unidentified
AYGGYGLGGGYGGYGVGVHSRYGVGGYGYGGLLGGYGLHY

SEQ ID NO: 174 - protein, 29 aa, unidentified
YGYGLAGYGGLYGGLHGAAYGLGGYGLHY

SEQ ID NO: 175 - protein, 33 aa, unidentified
LGYGLAGYGGLYGGLYGGHGLGGYGGVYGGYGL

SEQ ID NO: 176 - protein, 19 aa, unidentified
HGLHYLGFGGVLGYGGLHH

SEQ ID NO: 177 - protein, 33 aa, unidentified
GVYGLGHGAYGLGGYGGLHGAYGLGGYGGLYGG

SEQ ID NO: 178 - protein, 41 aa, unidentified
YGLGGYGALHGGLYGGYGLGGGLLYSYGGLVGGYGGLYHHA

SEQ ID NO: 179 - protein, 33 aa, unidentified
LFGGILGGYGGVLAGYGGLHHGAYGLGGYGGLY

SEQ ID NO: 180 - protein, 48 aa, unidentified
GGYGLGGYGLHGLHYLGFGGVLGYGGLHHGVYGLGGYGGLHGAYGLGG

SEQ ID NO: 181 - protein, 40 aa, unidentified
YGGLHGAYGLGGYGGLYGGTLSTLGYGYGGLLGGLGHAVG

SEQ ID NO: 182 - protein, 27 aa, unidentified
VGYGYGGLLGGYGGLYGGWGGVYGGLG

SEQ ID NO: 183 - protein, 21 aa, unidentified
VGYGYGGFLGGYGLGVYGHGY

SEQ ID NO: 184 - protein, 38 aa, unidentified
HGLHYLGFGGVLGYGGLHHGVYGLGGYGGLHGAYGLGG

SEQ ID NO: 185 - protein, 57 aa, unidentified
LYGGLHGAYGLGGYGGLYGGYGLGGYGALHGGLYGGYGLGGGGYGYGGLLGGYGLHY

SEQ ID NO: 186 - protein, 24 aa, unidentified
YGYGLAGYGGLYGGYGLGGYGLGY

SEQ ID NO: 187 - protein, 41 aa, unidentified
YGLGGFHGGYGLGGVGLGLGGFHGGYGFGGYGLGGFHGGYG

SEQ ID NO: 188 - protein, 33 aa, unidentified
VGFGGYGYGGIGGLYGGHYGGYGLGGAYGHYGG

SEQ ID NO: 189 - protein, 21 aa, unidentified
YGLGGGYGYGGLLGGLGHAVG

SEQ ID NO: 190 - protein, 26 aa, unidentified
GYGYGGLLGGYGGLYGGWGGVYGGLG

SEQ ID NO: 191 - protein, 26 aa, unidentified
LGYGGLLGGYGGLYGGYGLGGYGLGY

SEQ ID NO: 192 - protein, 17 aa, unidentified
YGYGLAGYGGLYGGLLH

SEQ ID NO: 193 - protein, 38 aa, unidentified
HGLHYLGFGGVLGYGGLHHGAYGLGGYGGLYGGYGLGG

SEQ ID NO: 194 - protein, 34 aa, unidentified
YGGLYGGYGALHGGYGLGYYGLAGYGGLYGGLLH

SEQ ID NO: 195 - protein, 21 aa, unidentified
TALGYGGLYGGYGLGAYGLGY

SEQ ID NO: 196 - protein, 26 aa, unidentified
LGYGGLLGGYGGLYGRYGVGGYGLGY

SEQ ID NO: 197 - protein, 19 aa, unidentified
GGYGSLLGGHGGLYGGLGL

SEQ ID NO: 198 - protein, 14 aa, unidentified
YGYGGVLGGYGQGL

SEQ ID NO: 199 - protein, 19 aa, unidentified
LGYGGLLGGYGGLHHGVYG

SEQ ID NO: 200 - protein, 33 aa, unidentified
GGYGGLYGGYGLGGYGGLHGAYGLGGYGGVYGG

SEQ ID NO: 201 - protein, 36 aa, unidentified
YGLGGHVGYGGYGYGGLGAYGHYGGYGLGGLYGGYG

SEQ ID NO: 202 - protein, 23 aa, unidentified
YGGLYGGYGLGGHVYGGYGLGGH

SEQ ID NO: 203 - protein, 33 aa, unidentified
VGYGGYGYGGGLYGGHYGGYGHFGGVHSHYGVG

SEQ ID NO: 204 - protein, 28 aa, unidentified
LGYGGLLGGYGALHGGLYGGYGLGGLHY

SEQ ID NO: 205 - protein, 330 aa, unidentified
MVGQSVSTVSHGVHAPSTGTLSYGYGGLFGGLFGGLGYGPVGQSVSTVSHGVHAPSTGTLSYGYGGLFGGLFGGLGYGPVGQSVSTVSHGVHAPSTGTLSYGYGGLFGGLFGGLGYGPVGQSVSTVSHGVHAPSTGTLSYGYGGLFGGLFGGLGYGPVGQSVSTVSHGVHAPSTGTLSYGYGGLFGGLFGGLGYGPVGQSVSTVSHGVHAPSTGTLSYGYGGLFGGLFGGLGYGPVGQSVSTVSHGVHAPSTGTLSYGYGGLFGGLFGGLGYGPVGQSVSTVSHGVHAPSTGTLSYGYGGLFGGLFGGLGYGPVGQSVSTVSHGVHAPTS

SEQ ID NO: 206 - protein, 305 aa, unidentified
MGTLSYGYGGLYGGLYGGLGYGPAAASVSTVHHPSTGTLSYGYGGLYGGLYGGLGYGPAAASVSTVHHPSTGTLSYGYGGLYGGLYGGLGYGPAAASVSTVHHPSTGTLSYGYGGLYGGLYGGLGYGPAAASVSTVHHPSTGTLSYGYGGLYGGLYGGLGYGPAAASVSTVHHPSTGTLSYGYGGLYGGLYGGLGYGPAAASVSTVHHPSTGTLSYGYGGLYGGLYGGLGYGPAAASVSTVHHPSTGTLSYGYGGLYGGLYGGLGYGPAAASVSTVHHPSTGTLSYGYGGLYGGLYGGLGYGPTS

SEQ ID NO: 207 - protein, 337 aa, unidentified
MGTLSYGYGGLYGGLYGGLGYGPVGQSVSTVSHGVHAPSTGTLSYGYGGLYGGLYGGLGYGPVGQSVSTVSHGVHAPSTGTLSYGYGGLYGGLYGGLGYGPVGQSVSTVSHGVHAPSTGTLSYGYGGLYGGLYGGLGYGPVGQSVSTVSHGVHAPSTGTLSYGYGGLYGGLYGGLGYGPVGQSVSTVSHGVHAPSTGTLSYGYGGLYGGLYGGLGYGPVGQSVSTVSHGVHAPSTGTLSYGYGGLYGGLYGGLGYGPVGQSVSTVSHGVHAPSTGTLSYGYGGLYGGLYGGLGYGPVGQSVSTVSHGVHAPSTGTLSYGYGGLYGGLYGGLGYGPTS

SEQ ID NO: 208 - protein, 330 aa, unidentified
MVGQSVSTVSHGVHAPSTGTLSYGYGGLYGGLYGGLGYGPVGQSVSTVSHGVHAPSTGTLSYGYGGLYGGLYGGLGYGPVGQSVSTVSHGVHAPSTGTLSYGYGGLYGGLYGGLGYGPVGQSVSTVSHGVHAPSTGTLSYGYGGLYGGLYGGLGYGPVGQSVSTVSHGVHAPSTGTLSYGYGGLYGGLYGGLGYGPVGQSVSTVSHGVHAPSTGTLSYGYGGLYGGLYGGLGYGPVGQSVSTVSHGVHAPSTGTLSYGYGGLYGGLYGGLGYGPVGQSVSTVSHGVHAPSTGTLSYGYGGLYGGLYGGLGYGPVGQSVSTVSHGVHAPTS

SEQ ID NO: 209 - DNA, 399 bp, unidentified
  source 1..399: mol_type = other DNA
gagtcagcgaccctctagaaataattttgtttaactttaagaaggagatataccatggtaggtcagagtgtttcgactgtctcgcacggagttcatgccccttctacagggacgttatcatatggatacggcggtttgtatggaggtctctacggtggattaggatatggacctgtcggtcaatcagtatctactgtgtcacatggggttcacgctccttcaactggtactcttagttatggttatgggggtctttatggaggactatatggcggattgggatatgggcctgttggtcaaagtgtatcaacagtttctcatggtgtccatgctccaactagttaacgcaggactggagcgctcgaggatccggctgctaacaaagcccgagcgagactc

SEQ ID NO: 210 - DNA, 399 bp, unidentified
  source 1..399: mol_type = other DNA
gagtcagcgaccctctagaaataattttgtttaactttaagaaggagatataccatggttggtcaaagtgtatcaacagtttctcatggtgtccatgctccaagcacaggaactttatcgtatgggtacgggggattatatggagggctctatggtgggttaggttacggtccggtaggacaatctgtaagtacagtgagccacggtgtacatgcacctagtactggaacattatcttatggctatggaggcttatacggaggtttatatggtggtctagggtatggtcctgtaggtcagagtgtttcgactgtctcgcacggagttcatgcccctactagttaacgcaggactggagcgctcgaggatccggctgctaacaaagcccgagcgagactc

SEQ ID NO: 211 - DNA, 396 bp, unidentified
  source 1..396: mol_type = other DNA
gagtcagcgaccctctagaaataattttgtttaactttaagaaggagatataccatgggaactttgtcttatggatatggcggtttatacggcggattgtatggaggtttgggatatggacctgcagcagctagtgttagcactgtacatcaccctagtacaggtacacttagttatggttacggaggtctatatgggggtctctacgggggtctcgggtatggtccggcagccgcgtcagtatctacagttcaccatccttcaacaggaacattatcttatggctatggagggctctatggtggtctttatggaggattaggatacggtcctactagttaacgcaggactggagcgctcgaggatccggctgctaacaaagcccgagcgagactc

SEQ ID NO: 212 - DNA, 396 bp, unidentified
  source 1..396: mol_type = other DNA
gagtcagcgaccctctagaaataattttgtttaactttaagaaggagatataccatgggaacattatcttatggctatggagggctctatggtggtctttatggaggattaggatacggtcctgccgctgcttctgtttctactgttcatcatccaagtactggtactctttcgtatgggtacggtggattatatggaggcttatatggtgggttaggttatgggccagctgcggcctctgtatcgactgtgcatcatccctcaactggaactttgtcttatggatatggcggtttatacggcggattgtatggaggtttgggatatggacctactagttaacgcaggactggagcgctcgaggatccggctgctaacaaagcccgagcgagactc

SEQ ID NO: 213 - DNA, 420 bp, unidentified
  source 1..420: mol_type = other DNA
gagtcagcgaccctctagaaataattttgtttaactttaagaaggagatataccatgggaactttgtcttatggatatggcggtttatacggcggattgtatggaggtttgggatatggacctgtaggtcagagtgtttcgactgtctcgcacggagttcatgcccctagtacaggtacacttagttatggttacggaggtctatatgggggtctctacgggggtctcgggtatggtccggtcggtcaatcagtatctactgtgtcacatggggttcacgctccttcaacaggaacattatcttatggctatggagggctctatggtggtctttatggaggattaggatacggtcctactagttaacgcaggactggagcgctcgaggatccggctgctaacaaagcccgagcgagactc

SEQ ID NO: 214 - DNA, 420 bp, unidentified
  source 1..420: mol_type = other DNA
gagtcagcgaccctctagaaataattttgtttaactttaagaaggagatataccatgggaacattatcttatggctatggagggctctatggtggtctttatggaggattaggatacggtcctgttggtcaaagtgtatcaacagtttctcatggtgtccatgctccaagtactggtactctttcgtatgggtacggtggattatatggaggcttatatggtgggttaggttatgggccagtaggacaatctgtaagtacagtgagccacggtgtacatgcaccttcaactggaactttgtcttatggatatggcggtttatacggcggattgtatggaggtttgggatatggacctactagttaacgcaggactggagcgctcgaggatccggctgctaacaaagcccgagcgagactc

SEQ ID NO: 215 - DNA, 399 bp, unidentified
  source 1..399: mol_type = other DNA
gagtcagcgaccctctagaaataattttgtttaactttaagaaggagatataccatggtaggtcagagtgtttcgactgtctcgcacggagttcatgccccttctacagggacgttatcatatggatacggcggtttgtttggaggtctcttcggtggattaggatatggacctgtcggtcaatcagtatctactgtgtcacatggggttcacgctccttcaactggtactcttagttatggttatgggggtctttttggaggactatttggcggattgggatatgggcctgttggtcaaagtgtatcaacagtttctcatggtgtccatgctccaactagttaacgcaggactggagcgctcgaggatccggctgctaacaaagcccgagcgagactc

SEQ ID NO: 216 - DNA, 399 bp, unidentified
  source 1..399: mol_type = other DNA
gagtcagcgaccctctagaaataattttgtttaactttaagaaggagatataccatggttggtcaaagtgtatcaacagtttctcatggtgtccatgctccaagcacaggaactttatcgtatgggtacgggggattatttggagggctctttggtgggttaggttacggtccggtaggacaatctgtaagtacagtgagccacggtgtacatgcacctagtactggaacattatcttatggctatggaggcttattcggaggtttatttggtggtctagggtatggtcctgtaggtcagagtgtttcgactgtctcgcacggagttcatgcccctactagttaacgcaggactggagcgctcgaggatccggctgctaacaaagcccgagcgagactc

SEQ ID NO: 217 - DNA, 20 bp, unidentified
  source 1..20: mol_type = other DNA
taatacgactcactataggg

SEQ ID NO: 218 - DNA, 19 bp, unidentified
  source 1..19: mol_type = other DNA
gctagttattgctcagcgg

(19) United States
(12) Patent Application Publication
(10) Pub. No.: US 2025/0145828 A1
(43) Pub. Date: May 8, 2025

(54) TRANSPARENT PROTEIN MATERIALS

(71) Applicant: TANDEM REPEAT TECHNOLOGIES, INC., Philadelphia, PA (US)

(72) Inventors: Benjamin D. ALLEN, Philadelphia, PA (US); Huihun JUNG, Philadelphia, PA (US)

(21) Appl. No.: 18/837,709

(22) PCT Filed: Feb. 9, 2023
(86) PCT No.: PCT/US2023/062296

§ 371 Date: Aug. 12, 2024

Related U.S. Application Data
(60) Provisional application No. 63/310,782, filed on Feb. 16, 2022.

(51) Int. Cl. C08L 89/00; C07K 14/435; C08J 5/18

(52) U.S. Cl. CPC C08L 89/00; C07K 14/43504; C08J 5/18; C08J 2389/00; C08L 2201/06; C08L 2201/10; C08L 2203/12; C08L 2203/16

(57) ABSTRACT

As can be seen, there are needs for protein materials that have desirable mechanical properties while maintaining optical transparency. The present embodiments are directed, in part, to adhesive coatings, films, and compositions comprising polypeptides such as, but not limited to, transparent adhesive coatings and films, and methods of making the same.

### CROSS-REFERENCE TO RELATED APPLICATIONS

This application claims benefit of U.S. Provisional Application No. 63/310,782, filed Feb. 16, 2022, which is hereby incorporated herein by reference in its entirety.

### SEQUENCE LISTING

This application contains a sequence listing filed in ST.26 format entitled “320020_2010_Sequence_Listing” created on Jan. 30, 2023. The content of the sequence listing is incorporated herein in its entirety.

### FIELD

Embodiments provided herein relate to adhesive coatings, films, and compositions comprising polypeptides such as, but not limited to, transparent adhesive coatings and films, and methods of making the same.

### BACKGROUND

Protein materials are ubiquitous in nature, playing critical protective and structural roles in forms as familiar as our own skin, hair, and fingernails, as well as providing the basis for some of our oldest technologies: fibers and textiles based on animal-derived materials like silk and wool. The development of modern biotechnology offers new possibilities for protein materials, including genetic engineering of a wide array of material properties, intrinsic biocompatibility and biodegradability, and sustainable, animal-free production in recombinant microbes. The most mature recombinant technology for protein-material production has been achieved for sequences based on various types of silk.

Recombinant silk-based sequences have been produced at scale and manufactured into a variety of products, including blended textiles, cosmetic additives, and coatings. However, silk-based sequences suffer from numerous drawbacks, including high molecular weights that stymie high-titer production, the difficulty of thermal manufacturing, and the limited tunability of mechanical properties. The recent introduction of recombinant materials based on squid-ring teeth (SRT) sequences has offered improvements to silk-based sequences, including lower molecular weight that enables high-titer production and simpler gene construction, thermal processability, and straightforward genetic tuning of mechanical properties. In addition to these benefits, SRT sequences demonstrate behaviors not observed in silks, including self-healing under mild conditions, directed self-assembly of non-biological materials into ordered nanomaterial composites, and hydration-switchable thermal conductivity. These desirable properties enable the future development of advanced devices, including those incorporating soft, flexible electronic and thermoelectric components.

Although the benefits of previously reported SRT-based material-forming polypeptide sequences are numerous, those sequences lack a critical property that would enable them to be used in optical coatings and electronics: optical transparency. Specifically, previously described SRT-based material designs are rendered opaque by the treatments that are used to develop their internal assembly states and hence their strength and flexibility. Said treatments include exposure to water and short-chain alcohols.

Furthermore, sustainable production requires that these materials be recyclable and derived from renewable feedstocks rather than petroleum. Production of existing synthetic polymer-based adhesives requires the consumption of finite resources and results in waste of valuable materials at device end-of-life. No existing material offers the required performance as well as renewable production and recyclability.

As can be seen, there are needs for protein materials that have desirable mechanical properties while maintaining optical transparency. The transparent adhesive coatings and compositions and methods of making the same, as described herein, fulfill these needs as well as others. Additionally, the transparent adhesive coatings and compositions as described herein can be produced by sustainable biomanufacturing without the use of fossil fuels or petroleum inputs and are recyclable.

### SUMMARY

Disclosed herein is a polypeptide that can be used to produce transparent materials. In some embodiments, the polypeptide has the formula:

A₁-(B₁-L₁-E₁-P₁)ₙ-B₁-G₁ Formula I,
wherein
A₁ is absent, is a methionine, or is an amino acid sequence 1 to 4 residues in length;
B₁ is an ASTVH-rich sequence amino acid sequence 6 to 17 residues in length comprising amino acids selected from the group consisting of alanine, serine, threonine, valine, histidine, glycine, glutamine, and proline, or any combination thereof.
L₁ is absent or is an amino acid sequence 1 to 7 residues in length comprising amino acids selected from the group consisting of proline, glycine, leucine, serine, and threonine, or any combination thereof;
E₁ is an GLY-rich amino acid sequence 8 to 58 residues in length comprising amino acids selected from the group consisting of glycine, leucine, tyrosine, phenylalanine, and proline, or any combination thereof;
P₁ is absent or is proline;
G₁ is absent or is an amino acid sequence 1 to 4 residues in length; and
wherein n is 4 to 100.

In particular embodiments, wherein E₁ is YGFGGLYGGLFGGLGFG (SEQ ID NO: 3) and B₁ comprises an amino acid sequence selected from the group consisting of SEQ ID NO: 11-88, or

In particular embodiments, wherein E₁ is YGYGGLFGGLFGGLGYG (SEQ ID NO: 2) and B₁ comprises an amino acid sequence selected from the group consisting of SEQ ID NO: 12, 13, 17, 19-39, 41-52, 54-59, 61, 64-68, 70-78, 80, 82-84, and 88, or

In particular embodiments, wherein E₁ is YGYGGLYGGLYGGLGYG (SEQ ID NO: 1) and B₁ comprises an amino acid sequence selected from the group consisting of SEQ ID NO: 12, 24, 27, 35, 37, 39, 55, 66, 67, 68, 71, 76, 82, and 83, or

In particular embodiments, wherein E₁ comprises an amino acid sequence selected from the group consisting of SEQ ID NO:90-204, B₁ comprises an amino acid sequence selected from the group consisting of SEQ ID NO: 13, 21-23, 25, 26, 29, 30, 32, 34, 36, 37, 39, 44-46, 48, 50-52, 55, 57, 58, 61, 64-68, 71, 72, 74, 76-78, 80-83, and 89, and L₁ is absent or is Pro.

In some embodiments, the polypeptide is a synthetic or recombinant supramolecular polypeptide.

In some embodiments, the A₁ is methionine (M). In some embodiments, L₁ is selected from the group consisting of SEQ ID NOs: 4 to 10. In some embodiments, G₁ is Thr-Ser (TS) or Pro-Thr-Ser (PTS). In some embodiments, n is 4-20. In some embodiments, A₁ is methionine (M), L₁ is SEQ ID NO:4, and G₁ is Pro-Thr-Ser (PTS). In some embodiments, E₁ is YGYGGLFGGLFGGLGYG (SEQ ID NO:2) and B₁ comprises is SEQ ID NO:23. For example, in some embodiments, the amino acid sequence is SEQ ID NO: 205.

Also disclosed is a composition comprising a disclosed polypeptide in a solvent. In some embodiments, the polypeptide is formulated as an adhesive or film. In some embodiments, the polypeptide is formulated as a fiber. In some embodiments, the solvent is dimethyl sulfoxide, formic acid, 1,1,1,3,3,3-hexafluoro-2-propanol, aqueous ammonia, aqueous alkali-metal hydroxide, or aqueous urea, In some embodiments, the solvent is an ionic liquid. In some embodiments, the solvent is 1-ethyl-3-methylimidazolium acetate.

In some embodiments, polypeptides, as described and provided for herein, are adhesive. In some embodiments, the polypeptide exhibits self-healing behavior. In some embodiments, the polypeptide is optically transparent. In some embodiments, the polypeptide shows superior transmission in the hydrated state. In some embodiments, the polypeptide shows superior transmission in the hydrated state in the optical region of the spectrum 400-700 nm.

In some embodiments, compositions comprise one or more polypeptides having a formula of Formula I as described and provided for herein. In some embodiments, compositions comprise one or more polypeptides having a formula of Formula I as described and provided for herein. In some embodiments, compositions comprise a polypeptide having a formula of Formula I as described and provided for herein.

In some embodiments, methods of making polypeptides having a formula of Formula I, are provided.

### DESCRIPTION OF DRAWINGS

FIGS. 1A and 1B show the architectures of two-block polypeptide sequences A and B, respectively. FIG. 1A shows a sequence architecture with GLY-rich termini and alternating GLY-rich and ASTVH-rich sequence blocks. FIG. 1B shows a sequence architecture with ASTVH-rich termini and alternating GLY-rich and ASTVH-rich sequence blocks.

FIG. 2 shows a first step of gene construction as described in Example 1.

FIG. 3 shows a second step of gene construction as described in Example 2.

FIG. 4 shows a polypeptide purification step as described in Example 5.

FIG. 5 shows transparency data for polypeptide sequences TR12n8 (SEQ ID NO: 206), TR18n8 (SEQ ID NO:207), TR8n8 (SEQ ID NO:208), and TR17n8 (SEQ ID NO: 205) in dry forms.

FIG. 6 shows transparency data for polypeptide sequences TR12n8, TR18n8, TR8n8, and TR17n8 in hydrated forms.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

As used herein, the term “about” means that the numerical value is approximate and small variations would not significantly affect the practice of the disclosed embodiments. Where a numerical limitation is used unless indicated otherwise by the context, “about” means the numerical value can vary by +10% and remain within the scope of the disclosed embodiments. Additionally, where a phrase recites “about x to y,” the term “about” modifies both x and y and can be used interchangeably with the phrase “about x to about y” unless context dictates differently.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps. Any polypeptide, composition, method, or step that uses the transitional phrase of “comprise” or “comprising” can also be said to describe the same with the transitional phase of “consisting of” or “consists.”

As used herein, “encode” or “encoding” refers to the inherent property of specific sequences of nucleotides in a polynucleotide, such as a gene, a cDNA, or an mRNA, to serve as templates for the synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

As used herein, “identity” as used herein refers to the subunit sequence identity between two polymeric molecules, such as between two nucleic acid or amino acid molecules, such as between two polynucleotides or polypeptide molecules. When two amino acid sequences have the same residues at the same positions, e.g., if a position in each of two polypeptide molecules is occupied by an Arginine, then they are identical at that position. The identity or extent to which two amino acids or two nucleic acid sequences have the same residues at the same positions in an alignment is often expressed as a percentage. The identity between two amino acid or two nucleic acid sequences is a direct function of the number of matching or identical positions; e.g., if half of the positions in two sequences are identical, the two sequences are 50% identical; if 90% of the positions (e.g., 9 of 10), are matched or identical, the two amino acids sequences are 90% identical.

As used herein, “PCR” or “polymerase chain reaction” refers to a method widely used to rapidly make millions to billions of copies (complete copies or partial copies) of a specific DNA sample, allowing scientists to take a very small sample of DNA and amplify it (or a part of it) to a large enough amount to study in detail.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). In some embodiments, such a sequence is at least 60%, 80%, 85%, 90%, or 95%, or even 99% identical at the amino acid level or nucleic acid to the sequence used for comparison. Other percentages of identity in reference to specific sequences are described herein.

Sequence identity can be measured/determined using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e3 and e100 indicating a closely related sequence. In some embodiments, sequence identity is determined by using BLAST with the default settings.

Provided for herein are adhesive coatings, films, and compositions comprising polypeptides. In some embodiment, the adhesive coating is transparent. In some embodiments, provided are two-block, amino-acid sequences of polypeptides that are optically transparent, adhesive, flexible, strong, and manufacturable and a method to produce such. The polypeptide sequences of this present disclosure exhibit an architecture reminiscent of block copolymers. This architecture comprises two alternating sequence blocks: one type of block, referred to as GLY-rich, consists primarily of the amino acids glycine, leucine, and tyrosine; the other type of block, referred to as ASTVH-rich, consists primarily of the amino acids alanine, serine, threonine, valine, and histidine. The composition rules of each block type are not strictly enforced; amino acids other than those listed are observed in each block type.

### Polypeptides

Disclosed herein are polypeptides having a formula of Formula I:

A₁-(B₁-L₁-E₁-P₁)ₙ-B₁-G₁ Formula I.

In some embodiments, A₁ is absent or methionine. In some embodiments, A₁ is absent. In some embodiments, A₁ is methionine. In some embodiments, A₁ is an amino acid sequence 1 to 4 amino acids in length.

In some embodiments, B₁ is an ASTVH-rich sequence amino acid sequence 6 to 17 residues in length comprising amino acids selected from the group consisting of alanine, serine, threonine, valine, histidine, glycine, glutamine, and proline, or any combination thereof. In some embodiments, B₁ is a first amino acid sequence comprising glycine. In some embodiments, B₁ is a first amino acid sequence comprising glutamine. In some embodiments, B₁ is a first amino acid sequence comprising serine. In some embodiments, B₁ is a first amino acid sequence comprising valine. In some embodiments, B₁ is a first amino acid sequence comprising threonine. In some embodiments, B₁ is a first amino acid sequence comprising histidine. In some embodiments, B₁ is a first amino acid sequence comprising alanine. In some embodiments, B₁ is a first amino acid sequence comprising proline. In some embodiments, B₁ is a first amino acid sequence comprising a combination of two or more of glycine, glutamine, serine, valine, threonine, histidine, alanine, and proline. In some embodiments, B₁ is a first amino acid sequence comprising glycine, glutamine, serine, valine, threonine, histidine, alanine, and proline.

The term ASTVH-rich sequence refers to a sequence that can comprise additional sequences and in a different order than a peptide of ASTVH. For example, in some embodiments, the ASTVH-rich sequence comprises at least one alanine, at least one serine, at least one threonine, at least one valine, and at least one histidine. In some embodiments, the ASTVH-rich sequence comprises two or more alanines. In some embodiments, the ASTVH-rich sequence comprises two or more serines. In some embodiments, the ASTVH-rich sequence comprises two or more threonines. In some embodiments, the ASTVH-rich sequence comprises two or more valines. In some embodiments, the ASTVH-rich sequence comprises two or more histidines.

In some embodiments, L₁ is absent or is an amino acid sequence 1 to 7 residues in length comprising amino acids selected from the group consisting of glycine, leucine, serine, and threonine, or any combination thereof. In some embodiments, L₁ is absent. In some embodiments, L₁ is a second amino sequence comprising glycine, leucine, serine and/or threonine. In some embodiments, L₁ is a second amino sequence comprising glycine, leucine, serine, or threonine. In some embodiments, L₁ is a second amino sequence comprising glycine, leucine, serine, and threonine. In some embodiments, L₁ is a second amino sequence comprising glycine. In some embodiments, L₁ is a second amino sequence comprising leucine. In some embodiments, L₁ is a second amino sequence comprising serine. In some embodiments, L₁ is selected from the group consisting of PSTGTLS (SEQ ID NO:4), PSTGTL (SEQ ID NO:5), PSTGT (SEQ ID NO:6), PSTG (SEQ ID NO:7), PST, PS, P, STGTLS (SEQ ID NO:8), STGTL (SEQ ID NO:9), STGT (SEQ ID NO: 10), STG, ST, and S.

In some embodiments, E₁ is an GLY-rich amino acid sequence 8 to 58 residues in length comprising amino acids selected from the group consisting of glycine, leucine, tyrosine, phenylalanine, and proline, or any combination thereof. In some embodiments, E₁ is a third amino sequence comprising glycine. In some embodiments, E₁ is a third amino sequence comprising leucine. In some embodiments, E₁ is a third amino sequence comprising tyrosine. In some embodiments, E₁ is a third amino sequence comprising phenylalanine. In some embodiments, E₁ is a third amino sequence comprising proline. In some embodiments, E₁ is a third amino sequence comprising a combination of two or more of glycine, leucine, tyrosine, phenylalanine, and proline. In some embodiments, E₁ is a third amino sequence comprising glycine, leucine, tyrosine, phenylalanine, and proline. In some embodiments, the GLY-rich sequence is YGYGGLYGGLYGGLGYG (SEQ ID NO: 1, GLY-rich-1), YGYGGLFGGLFGGLGYG (SEQ ID NO:2, GLY-rich-2), or YGFGGLYGGLFGGLGFG (SEQ ID NO:3).

In some embodiments, P₁ is absent or is a proline.

In some embodiments, G₁ is absent or is an amino acid sequence 1 to 4 residues in length. In some embodiments, G₁ is an amino acid sequence comprising serine and/or threonine. In some embodiments, G₁ is absent. In some embodiments, G₁ is an amino acid sequence comprising serine and/or threonine. In some embodiments, G₁ is an amino acid sequence comprising serine or threonine. In some embodiments, G₁ is an amino acid sequence comprising serine and threonine. In some embodiments, G₁ is an amino acid sequence comprising serine. In some embodiments, G₁ is an amino acid sequence comprising threonine.

In some embodiments, n is a range between 4-100. In some embodiments, n is 4-90. In some embodiments, n is 4-80. In some embodiments, n is 4-70. In some embodiments, n is 4-60. In some embodiments, n is 1-50. In some embodiments, n is 4-40. In some embodiments, n is 4-30. In some embodiments, n is 4-20. In some embodiments, n is 4-10. In some embodiments, n is 6-20. In some embodiments, n is 6-20. In some embodiments, n is 8-20. In some embodiments, n is 10-20. In some embodiments, n is 10-30. In some embodiments, n is 4-16. In some embodiments, n is 6-16. In some embodiments, n is 8-16. In some embodiments, n is 10-16. In some embodiments, n is 12-16. In some embodiments, n is 4-12. In some embodiments, n is 6-12. In some embodiments, n is 8-12. In some embodiments, n is 10-12. In some embodiments, n is 4. In some embodiments, n is 5. In some embodiments, n is 6. In some embodiments, n is 7. In some embodiments, n is 8. In some embodiments, n is 9. In some embodiments, n is 10. In some embodiments, n is 11. In some embodiments, n is 12. In some embodiments, n is 13. In some embodiments, n is 14. In some embodiments, n is 15. In some embodiments, n is 16. In some embodiments, n is 17. In some embodiments, n is 18. In some embodiments, n is 19. In some embodiments, n is 20.

In some embodiments, the polypeptide as described and provided for herein is a synthetic or recombinant supramolecular polypeptide. In some embodiments, the polypeptide as described and provided for herein is a synthetic supramolecular polypeptide. In some embodiments, the polypeptide as described and provided for herein is a recombinant supramolecular polypeptide.

In some embodiments, E₁ is YGFGGLYGGLFGGLGFG (SEQ ID NO:3) and B₁ is a naturally occurring sequence selected from the group consisting of AATAVHTTHHA (SEQ ID NO:11), VAHHSVVSRRYAI (SEQ ID NO: 12), SATAVSHTSH (SEQ ID NO:13), VGAAVSHVTHHA (SEQ ID NO: 14), HAVGAVSTLHH (SEQ ID NO:15), AAAVSHVTHHA (SEQ ID NO: 16), VATVTSQTSHHV (SEQ ID NO:17), AASAVSTSTH (SEQ ID NO:18), ASSAVSHTSHH (SEQ ID NO: 19), HSVAVGVHH (SEQ ID NO:20), HTVSHVSHG (SEQ ID NO: 21), VTSAVHTVS (SEQ ID NO:22), VGQSVSTVSHGVHA (SEQ ID NO:23), VAHHGTISRRYAI (SEQ ID NO:24), TGASVNTVSHGISHA (SEQ ID NO:25), VGASVSTVSHGIGH (SEQ ID NO:26), VGSTISHTTHGVHH (SEQ ID NO:27), AATSNSHTTHGVHH (SEQ ID NO:28), YYRKSVSTVSHGAHY (SEQ ID NO:29), HVGTSVHSVSHGA (SEQ ID NO:30), ATAVSHTTHHA (SEQ ID NO:31), VSSSVSHVSHGAHY (SEQ ID NO:32), VSSVRTVSHGLHH (SEQ ID NO:33), RSVSHTTHSA (SEQ ID NO:34), AVSTVSHGLGYGLHH (SEQ ID NO:35), YIGRSVSTVSHGSHY (SEQ ID NO:36), AVGHTTVTHAV (SEQ ID NO:37), AATTYRQTTHH (SEQ ID NO:38), YYRRSFSTVSHGAHY (SEQ ID NO:39), AATSVKTVSHGFH (SEQ ID NO:40), AATAVSPHNSS (SEQ ID NO:41), AATAVSHTTHGIHH (SEQ ID NO:42), AATTAVTHH (SEQ ID NO:43), HVGTSVHSVSHGV (SEQ ID NO:44), TGSSISTVSHGV (SEQ ID NO:45), VVSHVTHTI (SEQ ID NO: 46), AASSVTHTTHGVAH (SEQ ID NO:47), VTHYSHVSHDVHQ (SEQ ID NO: 48), AATTAVTQTHH (SEQ ID NO:49), MSSSVSHVSHTAHS (SEQ ID NO:50), ASTSVSHTTHSV (SEQ ID NO:51), TSVSQVSHTAHS (SEQ ID NO:52), GHAVTHTVHH (SEQ ID NO:53), AATTVSHTTHGAHH (SEQ ID NO:54), SSYYGRSASTVSHGTHY (SEQ ID NO:55), VSSVSTVSHGLHH (SEQ ID NO:56), HIGTSVSSVSHGA (SEQ ID NO: 57), HSVSHVSHG (SEQ ID NO:58), GAAFHY (SEQ ID NO:59), GVAAYSHSVHH (SEQ ID NO:60), VGASVSTVSHGVHA (SEQ ID NO:61), AATSVKTVSHGYH (SEQ ID NO: 62), ATASVSHTTHGVHH (SEQ ID NO:63), HAVSTVAHGIH (SEQ ID NO:64), AVSHVTHTI (SEQ ID NO:65), VRYHGYSIGH (SEQ ID NO:66), AVRHTTVTHAV (SEQ ID NO: 67), GATTYSHTTHAV (SEQ ID NO:68), VGGAVSTVHH (SEQ ID NO:69), AATTVSHSTHAV (SEQ ID NO:70), HASTTTHSIGL (SEQ ID NO:71), AVSHVTHTIPHA (SEQ ID NO:72), AAAVSHTTHHA (SEQ ID NO:73), TGSSISTVSHGVHS (SEQ ID NO: 74), VASSVSHTTHGVHH (SEQ ID NO:75), SAGGTTVSHSTHGV (SEQ ID NO:76), SVATRRVVY (SEQ ID NO:77), AGSSISTVSHGVHA (SEQ ID NO:78), AATSVSHTTHSV (SEQ ID NO:79), HSVSTVSHGA (SEQ ID NO:80), TGTSVSTVSHGV (SEQ ID NO:81), VIHGGATLSTVSHGV (SEQ ID NO:82), SHGVSHTAGYSSHY (SEQ ID NO: 83), VGSTSVSHTTHGVHH (SEQ ID NO:84), AATSYSHALHH (SEQ ID NO:85), AATTYSHTAHHA (SEQ ID NO:86), AATYSHTTHHA (SEQ ID NO:87), and GLLGAAATTYKHTTHHA (SEQ ID NO:88).

In some embodiments, E₁ is YGYGGLYGGLYGGLGYG (SEQ ID NO:1, GLY-rich-1) and B₁ is a naturally occurring sequence selected from the group consisting of VAHHSVVSRRYAI (SEQ ID NO:12), VAHHGTISRRYAI (SEQ ID NO:24), VGSTISHTTHGVHH (SEQ ID NO:27), AVSTVSHGLGYGLHH (SEQ ID NO:35), AVGHTTVTHAV (SEQ ID NO:37), YYRRSFSTVSHGAHY (SEQ ID NO:39), SSYYGRSASTVSHGTHY (SEQ ID NO:55), VRYHGYSIGH (SEQ ID NO:66), AVRHTTVTHAV (SEQ ID NO:67), GATTYSHTTHAV (SEQ ID NO:68), HASTTTHSIGL (SEQ ID NO:71), SAGGTTVSHSTHGV (SEQ ID NO:76), VIHGGATLSTVSHGV (SEQ ID NO: 82), and SHGVSHTAGYSSHY (SEQ ID NO:83).

In some embodiments, E₁ is YGYGGLFGGLFGGLGYG (SEQ ID NO:2, GLY-rich-2) and B₁ is a naturally occurring sequence selected from the group consisting of VAHHSVVSRRYAI (SEQ ID NO:12), SATAVSHTSH (SEQ ID NO:13), VATVTSQTSHHV (SEQ ID NO: 17), ASSAVSHTSHH (SEQ ID NO:19), HSVAVGVHH (SEQ ID NO:20), HTVSHVSHG (SEQ ID NO:21), VTSAVHTVS (SEQ ID NO:22), VGQSVSTVSHGVHA (SEQ ID NO:23), VAHHGTISRRYAI (SEQ ID NO:24), TGASVNTVSHGISHA (SEQ ID NO:25), VGASVSTVSHGIGH (SEQ ID NO:26), VGSTISHTTHGVHH (SEQ ID NO:27), AATSNSHTTHGVHH (SEQ ID NO:28), YYRKSVSTVSHGAHY (SEQ ID NO:29), HVGTSVHSVSHGA (SEQ ID NO:30), ATAVSHTTHHA (SEQ ID NO:31), VSSSVSHVSHGAHY (SEQ ID NO:32), VSSVRTVSHGLHH (SEQ ID NO:33), RSVSHTTHSA (SEQ ID NO:34), AVSTVSHGLGYGLHH (SEQ ID NO:35), YIGRSVSTVSHGSHY (SEQ ID NO:36), AVGHTTVTHAV (SEQ ID NO:37), AATTYRQTTHH (SEQ ID NO:38), YYRRSFSTVSHGAHY (SEQ ID NO:39), AATAVSPHNSS (SEQ ID NO:41), AATAVSHTTHGIHH (SEQ ID NO:42), AATTAVTHH (SEQ ID NO:43), HVGTSVHSVSHGV (SEQ ID NO:44), TGSSISTVSHGV (SEQ ID NO:45), VVSHVTHTI (SEQ ID NO:46), AASSVTHTTHGVAH (SEQ ID NO:47), VTHYSHVSHDVHQ (SEQ ID NO: 48), AATTAVTQTHH (SEQ ID NO:49), MSSSVSHVSHTAHS (SEQ ID NO:50), ASTSVSHTTHSV (SEQ ID NO:51), TSVSQVSHTAHS (SEQ ID NO:52), AATTVSHTTHGAHH (SEQ ID NO:54), SSYYGRSASTVSHGTHY (SEQ ID NO:55), VSSVSTVSHGLHH (SEQ ID NO:56), HIGTSVSSVSHGA (SEQ ID NO:57), HSVSHVSHG (SEQ ID NO:58), GAAFHY (SEQ ID NO:59), VGASVSTVSHGVHA (SEQ ID NO: 61), HAVSTVAHGIH (SEQ ID NO:64), AVSHVTHTI (SEQ ID NO:65), VRYHGYSIGH (SEQ ID NO:66), AVRHTTVTHAV (SEQ ID NO:67), GATTYSHTTHAV (SEQ ID NO:68), AATTVSHSTHAV (SEQ ID NO:70), HASTTTHSIGL (SEQ ID NO:71), AVSHVTHTIPHA (SEQ ID NO:72), AAAVSHTTHHA (SEQ ID NO:73), TGSSISTVSHGVHS (SEQ ID NO:74), VASSVSHTTHGVHH (SEQ ID NO:75), SAGGTTVSHSTHGV (SEQ ID NO:76), SVATRRVVY (SEQ ID NO:77), AGSSISTVSHGVHA (SEQ ID NO:78), HSVSTVSHGA (SEQ ID NO:80), VIHGGATLSTVSHGV (SEQ ID NO:82), SHGVSHTAGYSSHY (SEQ ID NO:83), VGSTSVSHTTHGVHH (SEQ ID NO:84), and GLLGAAATTYKHTTHHA (SEQ ID NO: 88).

In some embodiments, E₁ and B₁ are naturally occurring sequences. For example, in some embodiments, E₁ is selected from the group consisting of GYGLGGLYGGYGLGGLHYGGYGLGGLHYGGYGL (SEQ ID NO:90), HYGVGGLYGGYGLGGLHGGYGLGGIYGGYGAHY (SEQ ID NO:91), GVGGYGMGGLYGGYGLGGVYGGYGLGG (SEQ ID NO:92), GYGLGVGL (SEQ ID NO: 93), LGLGYGGYGLGLGYGLGHGYGLGLGAGI (SEQ ID NO:94), GLGLGYGYGLGHGLG (SEQ ID NO:95), GLGLGYGLGLGL (SEQ ID NO:96), MGGLYGGYGLGGVYGGYGLGGIYGGYGAHY (SEQ ID NO:97), GVGGLYGGYGLGGLYGGYGLGGLHGGYSLGGLY (SEQ ID NO:98), GGYGAHYGVGGLYGGYGLGGLHYGGYGLGGLHYGGYGLHY (SEQ ID NO:99), YGYGGLYGGLYGGLG (SEQ ID NO:100), VAYGGWGYGLGGLHGGWGYGLGGLHGGWGYALG (SEQ ID NO:101), GLYGGLHYVGLGYGGLYGGLHY (SEQ ID NO: 102), VGYGGFGLGFGGLYGGLHY (SEQ ID NO:103), SLGAYGGYGLGGLIGGHSVYH (SEQ ID NO:104), SLGAYGGYGLGGIVGGYGAYN (SEQ ID NO: 105), VGLGYGGFGLGYGGLYGGFGY (SEQ ID NO: 106), VAYGGLGYGFGF (SEQ ID NO:107), GYGGLYGGLGYHY (SEQ ID NO: 108), YGYGGLYGGLYGGLGY (SEQ ID NO: 109), VGYGGYGLGAYGAYGLGYGLHY (SEQ ID NO:110), VGYAGYGLG (SEQ ID NO:111), YGGFGYGLY (SEQ ID NO: 112), GYGGLYGHYGGYGLGGAYGH (SEQ ID NO:113), GIGGVYGHGIGGLGGVYGHGIGGVYGHGIGGLY (SEQ ID NO:114), GHGFGGAYGGYGGYGIGGVTYGGLGLGGLGYGGLGYGGLGYGGLGYGGLGY (SEQ ID NO: 115), GGLGYGGLGYGGLGAGGLYGGAVGLGYGLGGGYGGLYGLHL (SEQ ID NO: 116), ALGLGLYGGAHL (SEQ ID NO:117), GLGLNYGVYGLH (SEQ ID NO:118), GYGGWGYGLGGWGHGLGGLG (SEQ ID NO:119), YGGIGLGGLYGGYGAHF (SEQ ID NO: 120), HSVGWGLGGWGGYGLGYGVHA (SEQ ID NO:121), ALGAYGGYGFGGIVGGHSVYH (SEQ ID NO: 122), ALGGYGGYGLGGIVGG (SEQ ID NO: 123), ALGAYGGYGLGGLVGGFGAYH (SEQ ID NO: 124), VGFGGYGLGGYGLGGYGLGGYGLGGYGLGGLVG (SEQ ID NO:125), GYGSYHVGYGGYGLGGYGGYGLGGLTGGYGV (SEQ ID NO:126), GYGLGLGYGLGLGAG (SEQ ID NO: 127), LGLGYGYGLGLGYGLGLGAGI (SEQ ID NO: 128), HLGLGLGYGYGLGHGLG (SEQ ID NO: 129), GLGLGYGLGLGYGYGV (SEQ ID NO: 130), GYGLGLGLGGAGYGY (SEQ ID NO: 131), VGGYGGFGLGGYGGYGLGG (SEQ ID NO: 132), VGYGGLYGHYGGYGLGGVYGHGVGLGGVYGHGI (SEQ ID NO: 133), GGAYGGYGLGVGGLYGGYGGYGIGGVGGYGGFGLGGYGGYGLGG (SEQ ID NO: 134), VGYGGLYGHYGGYGLGGVYGHGVGLGGVYGHGV (SEQ ID NO: 135), GLGGVYSHGIGGAYGGYGLGVGGLYGGYGGYGIGG (SEQ ID NO:136), VLSGGLGLSGLSGGYGTYR (SEQ ID NO:137), GYGGVGYGGLGYGGLGYGVGGLYGLQY (SEQ ID NO:138), GYGGWGYGLGGWGHGLGGLGSYGLHY (SEQ ID NO: 139), HSVGWGLGGWGGYGLGYGVRS (SEQ ID NO: 140), YGDVYGGLYGGLYGGLLGA (SEQ ID NO: 141), VAYGGLGLGALGYGGLGYGGLGYGGLGAGGLYG (SEQ ID NO: 142), LHYGYGLGLGLYGAHL (SEQ ID NO:143), AYGGWGYSLGRWGQGLGGLGTYGLHY (SEQ ID NO:144), ALGGYGGYGLGGIVGGHSVYH (SEQ ID NO: 145), ALGEYGGYGLGGIVGGH (SEQ ID NO: 146), GFGGYGLGGYGLGGYGLGGYG (SEQ ID NO:147), IGFGGWGHGYGYSGLGFGGWGHGLGGWGHGYGY (SEQ ID NO:148), HAVGFGGWGHGIGLGHGFGY (SEQ ID NO:149), HAVGFGGWGHGFGY (SEQ ID NO: 150), HSVSYGGWGFGHGGLYGLH (SEQ ID NO: 151), HADYGVSGLGGYVSSY (SEQ ID NO:152), VGFGGYGLGGYGLGGYGLGGYGLGGYGLGGVVG (SEQ ID NO: 153), GFGGYHFGYGGVGYGGLGYGGLGYGVGGLYGLQY (SEQ ID NO: 154), VAYGGLGLGALGYGGLGYGGLGAGGLYGLHY (SEQ ID NO:155), AGLGYGLGGVYGGYGLHA (SEQ ID NO:156), YGYGGLYGGLGYHAGYGLGGYGLGYGLHY (SEQ ID NO: 157), VGWGLGGLYGGLHH (SEQ ID NO: 158), GYGGYGLGLGGLYGGLHY (SEQ ID NO:159), GYGGYGLGFGGLYGGFGY (SEQ ID NO: 160), AYGYGYGLGGYGGYGLYGGYGLHH (SEQ ID NO: 161), VAYGGWGYGLGGLHGGWGYGLGGLYGGLH (SEQ ID NO:162), VGYAGYGYGLGSYGGYAGLGLGLYGAGYHY (SEQ ID NO:163), YAYGGLYGGYGLGAYGY (SEQ ID NO:164), VGYAGYGYGLGAYGGYAGLGLGLYGAGYHY (SEQ ID NO:165), VGYGGFGLAGYGYGY (SEQ ID NO: 166), YGYGGLYGGYAGLGLGLYGAGYHY (SEQ ID NO: 167), VGYAGYGLGLYGAGYHY (SEQ ID NO: 168), VGYAGYGLGAYGGYAGYGLGAFGGYAGYGLGAF (SEQ ID NO: 169), GGYAGLGLGLYGAGYHYLGFGGLLGGYGGLHHGVYGLGGYGGLYGGYGLG (SEQ ID NO: 170), GYGLHGLHYLGFGGVLGYGGLHHGVYGLGGYGGLHGAYGLGG (SEQ ID NO: 171), YGGLHGAYGLGGYGGLYGGYGLGGHVGYGGYGYGGLGAYGHYGGYGLGGLYGGY GLGG (SEQ ID NO:172), AYGGYGLGGGYGGYGVGVHSRYGVGGYGYGGLLGGYGLHY (SEQ ID NO:173), YGYGLAGYGGLYGGLHGAAYGLGGYGLHY (SEQ ID NO:174), LGYGLAGYGGLYGGLYGGHGLGGYGGVYGGYGL (SEQ ID NO:175), HGLHYLGFGGVLGYGGLHH (SEQ ID NO:176), GVYGLGHGAYGLGGYGGLHGAYGLGGYGGLYGG (SEQ ID NO:177), YGLGGYGALHGGLYGGYGLGGGLLYSYGGLVGGYGGLYHHA (SEQ ID NO: 178), LEGGILGGYGGVLAGYGGLHHGAYGLGGYGGLY (SEQ ID NO:179), GGYGLGGYGLHGLHYLGFGGVLGYGGLHHGVYGLGGYGGLHGAYGLGG (SEQ ID NO: 180), YGGLHGAYGLGGYGGLYGGTLSTLGYGYGGLLGGLGHAVG (SEQ ID NO: 181), VGYGYGGLLGGYGGLYGGWGGVYGGLG (SEQ ID NO: 182), VGYGYGGFLGGYGLGVYGHGY (SEQ ID NO:183), HGLHYLGFGGVLGYGGLHHGVYGLGGYGGLHGAYGLGG (SEQ ID NO:184), LYGGLHGAYGLGGYGGLYGGYGLGGYGALHGGLYGGYGLGGGGYGYGGLLGGYGL HY (SEQ ID NO:185), YGYGLAGYGGLYGGYGLGGYGLGY (SEQ ID NO: 186), YGLGGFHGGYGLGGVGLGLGGFHGGYGFGGYGLGGFHGGYG (SEQ ID NO:187), VGFGGYGYGGIGGLYGGHYGGYGLGGAYGHYGG (SEQ ID NO:188), YGLGGGYGYGGLLGGLGHAVG (SEQ ID NO:189), GYGYGGLLGGYGGLYGGWGGVYGGLG (SEQ ID NO: 190), LGYGGLLGGYGGLYGGYGLGGYGLGY (SEQ ID NO: 191), YGYGLAGYGGLYGGLLH (SEQ ID NO: 192), HGLHYLGFGGVLGYGGLHHGAYGLGGYGGLYGGYGLGG (SEQ ID NO: 193), YGGLYGGYGALHGGYGLGYYGLAGYGGLYGGLLH (SEQ ID NO: 194), TALGYGGLYGGYGLGAYGLGY (SEQ ID NO:195), LGYGGLLGGYGGLYGRYGVGGYGLGY (SEQ ID NO:196), GGYGSLLGGHGGLYGGLGL (SEQ ID NO: 197), YGYGGVLGGYGQGL (SEQ ID NO: 198), LGYGGLLGGYGGLHHGVYG (SEQ ID NO: 199), GGYGGLYGGYGLGGYGGLHGAYGLGGYGGVYGG (SEQ ID NO:200), YGLGGHVGYGGYGYGGLGAYGHYGGYGLGGLYGGYG (SEQ ID NO:201), YGGLYGGYGLGGHVYGGYGLGGH (SEQ ID NO:202), VGYGGYGYGGGLYGGHYGGYGHFGGVHSHYGVG (SEQ ID NO:203), LGYGGLLGGYGALHGGLYGGYGLGGLHY (SEQ ID NO:204); and

B₁ is selected from the group consisting of SATAVSHTSH (SEQ ID NO:13), HTVSHVSHG (SEQ ID NO:21), VTSAVHTVS (SEQ ID NO:22), VGQSVSTVSHGVHA (SEQ ID NO:23), TGASVNTVSHGISHA (SEQ ID NO:25), VGASVSTVSHGIGH (SEQ ID NO: 26), YYRKSVSTVSHGAHY (SEQ ID NO:29), HVGTSVHSVSHGA (SEQ ID NO:30), VSSSVSHVSHGAHY (SEQ ID NO:32), RSVSHTTHSA (SEQ ID NO:34), YIGRSVSTVSHGSHY (SEQ ID NO:36), AVGHTTVTHAV (SEQ ID NO:37), YYRRSFSTVSHGAHY (SEQ ID NO:39), HVGTSVHSVSHGV (SEQ ID NO:44), TGSSISTVSHGV (SEQ ID NO:45), VVSHVTHTI (SEQ ID NO:46), VTHYSHVSHDVHQ (SEQ ID NO:48), MSSSVSHVSHTAHS (SEQ ID NO:50), ASTSVSHTTHSV (SEQ ID NO: 51), TSVSQVSHTAHS (SEQ ID NO:52), SSYYGRSASTVSHGTHY (SEQ ID NO: 55), HIGTSVSSVSHGA (SEQ ID NO:57), HSVSHVSHG (SEQ ID NO:58), VGASVSTVSHGVHA (SEQ ID NO:61), HAVSTVAHGIH (SEQ ID NO:64), AVSHVTHTI (SEQ ID NO:65), VRYHGYSIGH (SEQ ID NO:66), AVRHTTVTHAV (SEQ ID NO:67), GATTYSHTTHAV (SEQ ID NO:68), HASTTTHSIGL (SEQ ID NO:71), AVSHVTHTIPHA (SEQ ID NO:72), TGSSISTVSHGVHS (SEQ ID NO:74), SAGGTTVSHSTHGV (SEQ ID NO: 76), TGASVSTVSHGL (SEQ ID NO:89), SVATRRVVY (SEQ ID NO:77), AGSSISTVSHGVHA (SEQ ID NO:78), HSVSTVSHGA (SEQ ID NO:80), TGTSVSTVSHGV (SEQ ID NO:81), VIHGGATLSTVSHGV (SEQ ID NO:82), and SHGVSHTAGYSSHY (SEQ ID NO:83).

In some embodiments, polypeptides having a formula of Formula I as described and provided for herein are provided, wherein G₁ is Thr-Ser.

In some embodiments, the disclosed polypeptide has an amino acid sequence of MVGQSVSTVSHGVHAPSTGTLSYGYGGLFGGLFGGLGYGPVGQSVSTVSHGVHAPS TGTLSYGYGGLFGGLFGGLGYGPVGQSVSTVSHGVHAPSTGTLSYGYGGLFGGLFGG LGYGPVGQSVSTVSHGVHAPSTGTLSYGYGGLFGGLFGGLGYGPVGQSVSTVSHGV HAPSTGTLSYGYGGLFGGLFGGLGYGPVGQSVSTVSHGVHAPSTGTLSYGYGGLFGG LFGGLGYGPVGQSVSTVSHGVHAPSTGTLSYGYGGLFGGLFGGLGYGPVGQSVSTVS HGVHAPSTGTLSYGYGGLFGGLFGGLGYGPVGQSVSTVSHGVHAPTS (SEQ ID NO: 205, TR17n8), i.e. where A₁ is M, B₁ is VGQSVSTVSHGVHA (SEQ ID NO:23), L₁ is PSTGTLS (SEQ ID NO:4), E₁ is YGYGGLFGGLFGGLGYG (SEQ ID NO:2), P₁ is P, G₁ is PTS, and n is 8. In some embodiments, polypeptides substantially identical to SEQ ID NO: 205 are provided. In some embodiments, the polypeptide is at least, or about, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical as compared to SEQ ID NO:205.

In some embodiments, the disclosed polypeptide has an amino acid sequence

(SEQ ID NO: 206, TR12n8)
MGTLSYGYGGLYGGLYGGLGYGPAAASVSTVHHPSTGTLSYGYGGLYGG
LYGGLGYGPAAASVSTVHHPSTGTLSYGYGGLYGGLYGGLGYGPAAASV
STVHHPSTGTLSYGYGGLYGGLYGGLGYGPAAASVSTVHHPSTGTLSYG
YGGLYGGLYGGLGYGPAAASVSTVHHPSTGTLSYGYGGLYGGLYGGLGY
GPAAASVSTVHHPSTGTLSYGYGGLYGGLYGGLGYGPAAASVSTVHHPS
TGTLSYGYGGLYGGLYGGLGYGPAAASVSTVHHPSTGTLSYGYGGLYGG
LYGGLGYGPTS.

In some embodiments, the disclosed polypeptide has an amino acid sequence

(SEQ ID NO: 207, TR18n8)
MGTLSYGYGGLYGGLYGGLGYGPVGQSVSTVSHGVHAPSTGTLSYGYGG
LYGGLYGGLGYGPVGQSVSTVSHGVHAPSTGTLSYGYGGLYGGLYGGLG
YGPVGQSVSTVSHGVHAPSTGTLSYGYGGLYGGLYGGLGYGPVGQSVST
VSHGVHAPSTGTLSYGYGGLYGGLYGGLGYGPVGQSVSTVSHGVHAPST
GTLSYGYGGLYGGLYGGLGYGPVGQSVSTVSHGVHAPSTGTLSYGYGGL
YGGLYGGLGYGPVGQSVSTVSHGVHAPSTGTLSYGYGGLYGGLYGGLGY
GPVGQSVSTVSHGVHAPSTGTLSYGYGGLYGGLYGGLGYGPTS.

In some embodiments, the disclosed polypeptide has an amino acid sequence

(SEQ ID NO: 208, TR8n8)
MVGQSVSTVSHGVHAPSTGTLSYGYGGLYGGLYGGLGYGPVGQSVSTVS
HGVHAPSTGTLSYGYGGLYGGLYGGLGYGPVGQSVSTVSHGVHAPSTGT
LSYGYGGLYGGLYGGLGYGPVGQSVSTVSHGVHAPSTGTLSYGYGGLYG
GLYGGLGYGPVGQSVSTVSHGVHAPSTGTLSYGYGGLYGGLYGGLGYGP
VGQSVSTVSHGVHAPSTGTLSYGYGGLYGGLYGGLGYGPVGQSVSTVSH
GVHAPSTGTLSYGYGGLYGGLYGGLGYGPVGQSVSTVSHGVHAPSTGTL
SYGYGGLYGGLYGGLGYGPVGQSVSTVSHGVHAPTS.

In some embodiments, polypeptides having a formula of Formula I as described and provided for herein are provided, wherein the polypeptide is optically transparent.

In some embodiments, polypeptides having a formula of Formula I as described and provided for herein are provided, wherein the polypeptide shows superior transmission in the hydrated state.

In some embodiments, polypeptides having a formula of Formula I as described and provided for herein are provided, wherein the polypeptide shows superior transmission in the hydrated state in the optical region of the spectrum 400-700 nm.

In some embodiments, polypeptides having a formula of Formula I as described and provided for herein are provided, wherein the polypeptide is adhesive.

In some embodiments, polypeptides having a formula of Formula I as described and provided for herein are provided, wherein the polypeptide exhibits self-healing behavior.

In some embodiments, methods of making the disclosed polypeptides are provided. In some embodiments, the method comprises: a) selecting an ASTVH-rich sequence for B₁ and selecting a GLY-rich sequence for E₁; b) modifying the ASTVH-rich sequence selected in step a) by introducing one or more amino-acid substitutions, insertions, or deletions, and modifying the GLY-rich sequence selected in step a) by introducing one or more amino-acid substitutions, insertions, or deletions; c) forming a polypeptide sequence comprising at least four copies of the ASTVH-rich sequence and at least four copies of the GLY-rich sequence selected in step a), bearing any optional modifications introduced in step b); and d) optionally expressing recombinantly and purifying the polypeptide of step c), forming a test sample from the purified polypeptide, and confirming the material properties of said polypeptide, wherein the rest variables are defined and provided for herein. In some embodiments, no amino-acid substitutions, insertions, or deletions are introduced in step b). In some embodiments, no more than five substitutions, insertions, or deletions of individual amino acids are introduced in step b). In some embodiments, the polypeptide sequence of step c) comprises at least eight copies of the repeat-unit sequence B₁-L₁-E₁-P₁ selected in step a), bearing any optional modifications introduced in step b). In some embodiments, the polypeptide sequence of step c) comprises eight copies of the repeat-unit sequence B₁-L₁-E₁-P₁ selected in step a), bearing any optional modifications introduced in step b). In some embodiments, the recombinant expression of step d) is performed in a recombinant strain of E. coli. In some embodiments, at least one copy of the chosen and modified ASTVH-rich sequence is placed within five amino acids of each terminus of the polypeptide sequence. In some embodiments, the confirmed material properties of step d) comprise a plurality of elasticity, self-healing ability, transparency, or adhesion capability.

### Definitions

As used herein, “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

By the term “modified” as used herein, is meant a changed state or structure of a molecule or cell as provided herein. Molecules may be modified in many ways, including chemically, structurally, and functionally, such as mutations, substitutions, insertions, or deletions (e.g. internal deletions or truncations). Cells may be modified through the introduction of nucleic acids or the expression of heterologous proteins.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some versions contain an intron(s).

The term “polynucleotide” as used herein is defined as a chain of nucleotides. Furthermore, nucleic acids are polymers of nucleotides. Thus, the terms “nucleic acids” and “polynucleotides” as used herein are interchangeable. As used herein, polynucleotides include but are not limited to, all nucleic acid sequences which are obtained by any methods available in the art, including, without limitation, recombinant methods, i.e., the cloning of nucleic acid sequences from a recombinant library or a cell genome, using cloning technology and PCR, and the like, and by synthetic means.

As used herein, the terms “peptide,” “polypeptide,” and “protein” are used interchangeably, and refer to a compound comprised of a plurality of amino acid residues covalently linked by peptide bonds. As used herein, the term refers to both short chains, which also commonly are referred to in the art as peptides, oligopeptides, and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

### SPECIFIC EMBODIMENTS

Embodiment 1. A polypeptide having a formula:

A₁-(B₁-L₁-E₁-P₁)ₙ-B₁-G₁ Formula I,
wherein
A₁ is absent, is a methionine, or is an amino acid sequence 1 to 4 residues in length;
B₁ is an ASTVH-rich sequence amino acid sequence 6 to 17 residues in length comprising amino acids selected from the group consisting of alanine, serine, threonine, valine, histidine, glycine, glutamine, and proline, or any combination thereof.
L₁ is absent or is an amino acid sequence 1 to 7 residues in length comprising amino acids selected from the group consisting of proline, glycine, leucine, serine, and threonine, or any combination thereof;
E₁ is an GLY-rich amino acid sequence 8 to 58 residues in length comprising amino acids selected from the group consisting of glycine, leucine, tyrosine, phenylalanine, and proline, or any combination thereof;
P₁ is absent or is proline;
G₁ is absent or is an amino acid sequence 1 to 4 residues in length;
wherein n is 4 to 100; and
(i) wherein E₁ is YGFGGLYGGLFGGLGFG (SEQ ID NO:3) and B₁ comprises an amino acid sequence selected from the group consisting of SEQ ID NO: 11-88, or
(ii) wherein E₁ is YGYGGLFGGLFGGLGYG (SEQ ID NO:2) and B₁ comprises an amino acid sequence selected from the group consisting of SEQ ID NO: 12, 13, 17, 19-39, 41-52, 54-59, 61, 64-68, 70-78, 80, 82-84, and 88, or
(iii) wherein E₁ is YGYGGLYGGLYGGLGYG (SEQ ID NO:1) and B₁ comprises an amino acid sequence selected from the group consisting of SEQ ID NO: 12, 24, 27, 35, 37, 39, 55, 66, 67, 68, 71, 76, 82, and 83, or
(iv) wherein E₁ comprises an amino acid sequence selected from the group consisting of SEQ ID NO:90-204, B₁ comprises an amino acid sequence selected from the group consisting of SEQ ID NO:13, 21-23, 25, 26, 29, 30, 32, 34, 36, 37, 39, 44-46, 48, 50-52, 55, 57, 58, 61, 64-68, 71, 72, 74, 76-78, 80-83, and 89, and L₁ is absent or is Pro.

Embodiment 2. The polypeptide of embodiment 1, wherein the polypeptide is a synthetic or recombinant supramolecular polypeptide.

Embodiment 3. The polypeptide of embodiment 1 or 2, wherein the A₁ is methionine (M).

Embodiment 4. The polypeptide of any one of embodiments 1 to 3, wherein L₁ is selected from the group consisting of SEQ ID NOs: 4 to 10.

Embodiment 5. The polypeptide of any one of embodiments 1 to 4, wherein G₁ is Thr-Ser (TS) or Pro-Thr-Ser (PTS).

Embodiment 6. The polypeptide of any one of embodiments 1 to 5, wherein n is 4-20.

Embodiment 7. The polypeptide of claim 1, wherein A₁ is methionine (M), L₁ is SEQ ID NO:4, and G₁ is Pro-Thr-Ser (PTS).

Embodiment 8. The polypeptide of any one of embodiments to 1 to 7, wherein E₁ is YGYGGLFGGLFGGLGYG (SEQ ID NO:2) and B₁ comprises is SEQ ID NO:23.

Embodiment 9. The polypeptide of embodiment 8 comprising the amino acid sequence SEQ ID NO:205.

Embodiment 10. A composition comprising a polypeptide of any one of embodiments 1 to 9 in a solvent.

Embodiment 11. The composition of embodiment 10, wherein the polypeptide is formulated as an adhesive or film.

Embodiment 12. The composition of embodiment 10, wherein the polypeptide is formulated as a fiber.

Embodiment 13. The composition of any one of embodiments 10 to 12, wherein the solvent is dimethyl sulfoxide, formic acid, 1,1,1,3,3,3-hexafluoro-2-propanol, aqueous ammonia, aqueous alkali-metal hydroxide, aqueous urea,

Embodiment 14. The composition of any one of embodiments 10 to 12, wherein the solvent is an ionic liquid.

Embodiment 15. The composition of embodiment 14, wherein the solvent is 1-ethyl-3-methylimidazolium acetate.

Although the present embodiments have been described in connection with certain specific embodiments for instructional purposes, the present embodiments are not limited thereto. Accordingly, various modifications, adaptations, and combinations of various features of the described embodiments can be practiced without departing from the scope of the invention as set forth in the claims. Furthermore, the following examples are illustrative, but not limiting, of the compounds, compositions and methods described herein. Other suitable modifications and adaptations known to those skilled in the art are within the scope of the following embodiments. Any and all journal articles, patent applications, issued patents, or other cited references are incorporated by reference in their entirety.

### EXAMPLES

### Example 1: Building Plasmid pET-14b-TR8n4

Example 1 provides methods of making polypeptide pET-14b-TR8n4 as described herein. A pET-system expression constructed to produce the polypeptide TR8n4 was prepared as follows:

1. Obtained double-stranded DNA fragments with sequences TR8_1-2 and TR8_3-4 (SEQ ID NO:209 and SEQ ID NO:210).

(SEQ ID NO: 209, TR8_1-2)
GAGTCAGCGACCCTCTAGAAATAATTTTGTTTAACTTTAAGAAGGAGAT
ATACCATGGTAGGTCAGAGTGTTTCGACTGTCTCGCACGGAGTTCATGC
CCCTTCTACAGGGACGTTATCATATGGATACGGCGGTTTGTATGGAGGT
CTCTACGGTGGATTAGGATATGGACCTGTCGGTCAATCAGTATCTACTG
TGTCACATGGGGTTCACGCTCCTTCAACTGGTACTCTTAGTTATGGTTA
TGGGGGTCTTTATGGAGGACTATATGGCGGATTGGGATATGGGCCTGTT
GGTCAAAGTGTATCAACAGTTTCTCATGGTGTCCATGCTCCAACTAGTT
AACGCAGGACTGGAGCGCTCGAGGATCCGGCTGCTAACAAAGCCCGAGC
GAGACTC.
(SEQ ID NO: 210, TR8_3-4)
GAGTCAGCGACCCTCTAGAAATAATTTTGTTTAACTTTAAGAAGGAGAT
ATACCATGGTTGGTCAAAGTGTATCAACAGTTTCTCATGGTGTCCATGC
TCCAAGCACAGGAACTTTATCGTATGGGTACGGGGGATTATATGGAGGG
CTCTATGGTGGGTTAGGTTACGGTCCGGTAGGACAATCTGTAAGTACAG
TGAGCCACGGTGTACATGCACCTAGTACTGGAACATTATCTTATGGCTA
TGGAGGCTTATACGGAGGTTTATATGGTGGTCTAGGGTATGGTCCTGTA
GGTCAGAGTGTTTCGACTGTCTCGCACGGAGTTCATGCCCCTACTAGTT
AACGCAGGACTGGAGCGCTCGAGGATCCGGCTGCTAACAAAGCCCGAGC
GAGACTC.

For example, such fragments can be ordered from a commercial DNA synthesis provider, for example, from Twist Bioscience.

2. Obtained a sample of plasmid vector pET-14b, for example, from EMD Millipore™.

3. Set up three separate digestions as follows:
a. Vector digestion. In a first 200-μL PCR tube, combined were the following:
i. 19.5 μL ultrapure water
ii. 2 μL pET-14b (500 ng/μL)
iii. 2.5 μL 10× Cutsmart buffer (New England Biolabs)
iv. 0.5 UL Xhol (20 units/μL, New England Biolabs)
v. 0.5 μL NcoI-HF (20 units/μL, New England Biolabs)
b. Fragment 1 digestion. In a second 200-μL PCR tube, combined were the following:
i. 4.5 μL ultrapure water
ii. 4 μL DNA fragment TR8_1-2 (lyophilized powder resuspended to 10 ng/μL in ultrapure water)
iii. 1 μL 10× Cutsmart Buffer (New England Biolabs)
iv. 0.25 μL Mlyl (10 units/μL, New England Biolabs)
v. 0.25 μL SpeI-HF (20 units/μL, New England Biolabs)
c. Fragment 2 digestion. In a third 200-μL PCR tube, combined were the following:
i. 4.5 μL ultrapure water
ii. 4 μL DNA fragment TR8_3-4 (lyophilized powder resuspended to 10 ng/μL in ultrapure water)
iii. 1 μL 10× Cutsmart Buffer (New England Biolabs)
iv. 0.25 μL Mlyl (10 units/μL, New England Biolabs)
v. 0.25 μL NcoI-HF (20 units/μL, New England Biolabs)
d. In a thermocycler, PCR machine, or similar device, each tube was incubated at 37° C. for 1 hour, followed by 80° C. for 20 minutes to heat-kill the enzymes.

4. Assembled the two digested fragments into the digested vector as follows:
a. In a 200-μL PCR tube, combined were the following:
i. 3 μL 2×NEB HiFi Assembly Master Mix (New England Biolabs)
ii. 1 μL heat-killed pET-14b vector digestion
iii. 1 μL heat-killed TR8_1-2 fragment digestion
iv. 1 μL heat-killed TR8_3-4 fragment digestion
b. In a thermocycler, PCR machine, or similar device, incubated the tube at 50° C. for 15 minutes.

5. Transformed the assembly mixture into competent E. coli cells with the following steps. Following the manufacturer's protocol, 5 μL of the assembly mixture was added into one aliquot of ice-thawed Mix & Go! Competent Cells-Zymo 10B cells (Zymo Research) or the like, mixed by flicking the tube gently, incubated on ice for 5 minutes, and spread the mixture onto an LB/agar plate (tryptone 10 g/L, yeast extract 5 g/L, NaCl 10 g/L, agar 15 g/L), supplemented with 100 μg/mL carbenicillin, that had been prewarmed to 37° C. The resulting plate was incubated at 37° C. for 14-18 hours until distinct colonies were visible. As will be familiar to one skilled in the art, a variety of E. coli strains, competent-cell protocols, and transformation protocols can be alternatively applied during this step. Acceptable strains include, but are not limited to, DH5α, DH10β, and XL1-Blue. Acceptable transformation approaches include, but are not limited to, heat shock and electroporation.

6. Screened colonies for the desired insert sequence with the following steps. 4-8 individual colonies were picked and transferred into individual 4-mL LB media cultures (tryptone 10 g/L, yeast extract 5 g/L, NaCl 10 g/L) supplemented with 200 μg/mL carbenicillin in 14-mL disposable culture tubes. The culture tubes were incubated at 37° C. and 200 rpm for 12-16 hours, until turbid. Plasmid DNA was isolated from each culture using the ZymoPURE Plasmid Miniprep Kit (Zymo Research) or the like, according to the manufacturer's protocol, or substituted any other protocol for plasmid isolation from E. coli culture. Each plasmid sample was analyzed by Sanger sequencing using a commercial service provider (e.g., Genewiz, Inc.) using the T7 and T7 Terminator primers (SEQ ID NO:217 and SEQ ID NO:218).

FIG. 2 shows the first step of gene construction as described herein. Synthetic DNA fragments and destination vector, digested with restriction enzymes, were assembled using NEB HiFi Assembly into an expression vector for the n=4 polypeptide. Each digested DNA bore overlap regions at each end that allowed NEB HiFi Assembly with its partner DNAs. F1: fragment 1, containing two repeat-unit coding sequences. F2: fragment 2, containing two more repeat-unit coding sequences. P: The promoter region of the expression vector. T: The terminator region of the expression vector.

### Example 2: Building Plasmid pET-14b-TR8n8

Example 2 provides methods of making the polypeptide sequence TR8n8 (SEQ ID NO: 208) as described herein. With a sequence-verified plasmid sample for pET-14b-TR8n4 prepared according to the methods as described and provided for in Example 1, the polypeptide sequence TR8n8 (SEQ ID NO:208) was prepared as follows:

1. Set up two separate digestions as follows:
a. Insert digestion. In a first 200-μL PCR tube, combined were the following:
i. 4.5 μL ultrapure water
ii. 4 μL pET-14b-TR8n4 plasmid (50 ng/μL, built and sequence-verified as described in “Building pET-14b-TR8n4” above)
iii. 1 μL 10× Cutsmart buffer (New England Biolabs)
iv. 0.25 μL Xhol (20 units/μL, New England Biolabs)
v. 0.25 μL NcoI-HF (20 units/μL, New England Biolabs)
b. Vector digestion. In a second 200-μL PCR tube, combined were the following:
i. 4.75 μL ultrapure water
ii. 4 μL pET-14b-TR8n4 plasmid (50 ng/μL, built and sequence-verified as described in “Building pET-14b-TR8n4” above)
iii. 1 μL 10× Cutsmart Buffer (New England Biolabs)
iv. 0.25 μL SpeI-HF (20 units/μL, New England Biolabs)
c. In a thermocycler, PCR machine, or similar device, each tube was incubated at 37° C. for 1 hour, followed by 80° C. for 20 minutes to heat-kill the enzymes.

2. Assembled the digested insert and digested vector as follows:
a. In a 200-μL PCR tube, combined were the following:
i. 3 μL 2×NEB HiFi Assembly Master Mix (New England Biolabs)
ii. 1.5 μL heat-killed pET-14b-TR8n4 vector digestion
iii. 1.5 μL heat-killed pET-14b-TR8n4 insert digestion
b. In a thermocycler, PCR machine, or similar device, the tube was incubated at 50° C. for 15 minutes.

3. The assembly mixture was transformed into competent E. coli cells with the following steps. Following the manufacturer's protocol, 5 μL of the assembly mixture was added into one aliquot of ice-thawed Mix & Go! Competent Cells-Zymo 10B cells (Zymo Research) or the like, mixed by flicking the tube gently, incubated on ice for 5 minutes, and the mixture was spread onto an LB/agar plate (tryptone 10 g/L, yeast extract 5 g/L, NaCl 10 g/L, agar 15 g/L), supplemented with 100 μg/mL carbenicillin, that had been prewarmed to 37° C. The resulting plate was incubated at 37° C. for 14-18 hours until distinct colonies were visible. As will be familiar to one skilled in the art, a variety of E. coli strains, competent-cell protocols, and transformation protocols can be alternatively applied during this step. Acceptable strains include, but are not limited to, DH5α, DH10β, and XL1-Blue. Acceptable transformation approaches include, but are not limited to, heat shock and electroporation.

4. Colonies were screened for the desired insert sequence with the following steps. 4-8 individual colonies were picked and transferred into individual 4-mL LB media cultures (tryptone 10 g/L, yeast extract 5 g/L, NaCl 10 g/L) supplemented with 200 μg/mL carbenicillin in 14-mL disposable culture tubes. The culture tubes were incubated at 37° C. and 200 rpm for 12-16 hours until turbid. Plasmid DNA was isolated from each culture using the ZymoPURE Plasmid Miniprep Kit (Zymo Research) or the like, according to the manufacturer's protocol, or substitute any other protocol for plasmid isolation from E. coli culture. Each plasmid sample was analyzed by Sanger sequencing using a commercial service provider (e.g., Genewiz, Inc.) using the T7 and T7 Terminator primers (SEQ ID NO:217 and SEQ ID NO:218).

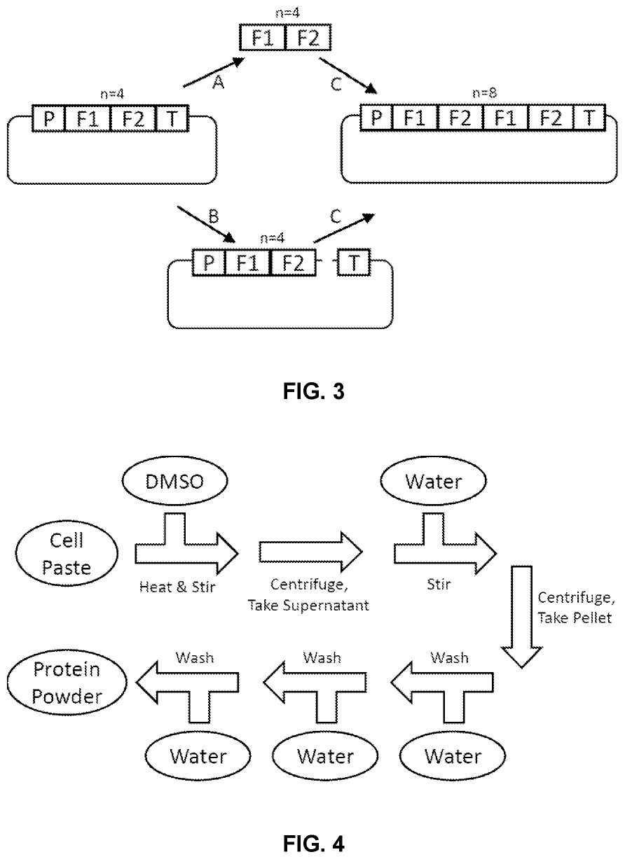

FIG. 3 shows a second step of gene construction as described herein. The n=4 construct from the first step was used to build the n=8 construct. A: The n=4 construct was digested to liberate an n=4 coding sequence DNA. B: The same n=4 construct was digested to open the circular DNA and expose compatible ends for NEB HiFi Assembly with the DNA from A. C: The DNAs produced in steps A and B were combined and assembled into a complete expression vector for the n=8 polypeptide.

### Example 3: Building Plasmids pET-14b-TR12n8, pET-14b-TR17n8, and pET-14b-TR18n8 and their Variants

Example 3 provides methods for making polypeptide sequences TR12n8 (SEQ ID NO: 206), TR18n8 (SEQ ID NO:207), TR17n8 (SEQ ID NO:205) and their variants. As described and provided for herein, these polypeptide sequences were prepared according to the steps described in Examples 1 and 2 by substituting appropriate synthetic double-stranded DNA fragments as described herein. Specifically, pET-14b-TR12n8 was built by applying the same protocol by using DNA fragments TR12_1-2 (SEQ ID NO:211) and TR12_3-4 (SEQ ID NO:212). Likewise, pET-14b-TR18n8 was built by applying the same protocol by using DNA fragments TR18_1-2 (SEQ ID NO: 213) and TR18_3-4 (SEQ ID NO:214), while pET-14b-TR17n8 was built by applying the same protocol by using DNA fragments TR17_1-2 (SEQ ID NO:215) and TR17_3-4 (SEQ ID NO:216).

(SEQ ID NO: 211, TR12_1-2)
GAGTCAGCGACCCTCTAGAAATAATTTTGTTTAACTTTAAGAAGGAGAT
ATACCATGGGAACTTTGTCTTATGGATATGGCGGTTTATACGGCGGATT
GTATGGAGGTTTGGGATATGGACCTGCAGCAGCTAGTGTTAGCACTGTA
CATCACCCTAGTACAGGTACACTTAGTTATGGTTACGGAGGTCTATATG
GGGGTCTCTACGGGGGTCTCGGGTATGGTCCGGCAGCCGCGTCAGTATC
TACAGTTCACCATCCTTCAACAGGAACATTATCTTATGGCTATGGAGGG
CTCTATGGTGGTCTTTATGGAGGATTAGGATACGGTCCTACTAGTTAAC
GCAGGACTGGAGCGCTCGAGGATCCGGCTGCTAACAAAGCCCGAGCGAG
ACTC
(SEQ ID NO: 212, TR12_3-4)
GAGTCAGCGACCCTCTAGAAATAATTTTGTTTAACTTTAAGAAGGAGAT
ATACCATGGGAACATTATCTTATGGCTATGGAGGGCTCTATGGTGGTCT
TTATGGAGGATTAGGATACGGTCCTGCCGCTGCTTCTGTTTCTACTGTT
CATCATCCAAGTACTGGTACTCTTTCGTATGGGTACGGTGGATTATATG
GAGGCTTATATGGTGGGTTAGGTTATGGGCCAGCTGCGGCCTCTGTATC
GACTGTGCATCATCCCTCAACTGGAACTTTGTCTTATGGATATGGCGGT
TTATACGGCGGATTGTATGGAGGTTTGGGATATGGACCTACTAGTTAAC
GCAGGACTGGAGCGCTCGAGGATCCGGCTGCTAACAAAGCCCGAGCGAG
ACTC.
(SEQ ID NO: 213, TR18_1-2)
GAGTCAGCGACCCTCTAGAAATAATTTTGTTTAACTTTAAGAAGGAGAT
ATACCATGGGAACTTTGTCTTATGGATATGGCGGTTTATACGGCGGATT
GTATGGAGGTTTGGGATATGGACCTGTAGGTCAGAGTGTTTCGACTGTC
TCGCACGGAGTTCATGCCCCTAGTACAGGTACACTTAGTTATGGTTACG
GAGGTCTATATGGGGGTCTCTACGGGGGTCTCGGGTATGGTCCGGTCGG
TCAATCAGTATCTACTGTGTCACATGGGGTTCACGCTCCTTCAACAGGA
ACATTATCTTATGGCTATGGAGGGCTCTATGGTGGTCTTTATGGAGGAT
TAGGATACGGTCCTACTAGTTAACGCAGGACTGGAGCGCTCGAGGATCC
GGCTGCTAACAAAGCCCGAGCGAGACTC
(SEQ ID NO: 214, TR18_3-4)
GAGTCAGCGACCCTCTAGAAATAATTTTGTTTAACTTTAAGAAGGAGAT
ATACCATGGGAACATTATCTTATGGCTATGGAGGGCTCTATGGTGGTCT
TTATGGAGGATTAGGATACGGTCCTGTTGGTCAAAGTGTATCAACAGTT
TCTCATGGTGTCCATGCTCCAAGTACTGGTACTCTTTCGTATGGGTACG
GTGGATTATATGGAGGCTTATATGGTGGGTTAGGTTATGGGCCAGTAGG
ACAATCTGTAAGTACAGTGAGCCACGGTGTACATGCACCTTCAACTGGA
ACTTTGTCTTATGGATATGGCGGTTTATACGGCGGATTGTATGGAGGTT
TGGGATATGGACCTACTAGTTAACGCAGGACTGGAGCGCTCGAGGATCC
GGCTGCTAACAAAGCCCGAGCGAGACTC.
(SEQ ID NO: 215, TR17_1-2)
GAGTCAGCGACCCTCTAGAAATAATTTTGTTTAACTTTAAGAAGGAGAT
ATACCATGGTAGGTCAGAGTGTTTCGACTGTCTCGCACGGAGTTCATGC
CCCTTCTACAGGGACGTTATCATATGGATACGGCGGTTTGTTTGGAGGT
CTCTTCGGTGGATTAGGATATGGACCTGTCGGTCAATCAGTATCTACTG
TGTCACATGGGGTTCACGCTCCTTCAACTGGTACTCTTAGTTATGGTTA
TGGGGGTCTTTTTGGAGGACTATTTGGCGGATTGGGATATGGGCCTGTT
GGTCAAAGTGTATCAACAGTTTCTCATGGTGTCCATGCTCCAACTAGTT
AACGCAGGACTGGAGCGCTCGAGGATCCGGCTGCTAACAAAGCCCGAGC
GAGACTC.
(SEQ ID NO: 216, TR17_3-4)
GAGTCAGCGACCCTCTAGAAATAATTTTGTTTAACTTTAAGAAGGAGAT
ATACCATGGTTGGTCAAAGTGTATCAACAGTTTCTCATGGTGTCCATGC
TCCAAGCACAGGAACTTTATCGTATGGGTACGGGGGATTATTTGGAGGG
CTCTTTGGTGGGTTAGGTTACGGTCCGGTAGGACAATCTGTAAGTACAG
TGAGCCACGGTGTACATGCACCTAGTACTGGAACATTATCTTATGGCTA
TGGAGGCTTATTCGGAGGTTTATTTGGTGGTCTAGGGTATGGTCCTGTA
GGTCAGAGTGTTTCGACTGTCTCGCACGGAGTTCATGCCCCTACTAGTT
AACGCAGGACTGGAGCGCTCGAGGATCCGGCTGCTAACAAAGCCCGAGC
GAGACTC.

Variants of polypeptide sequences TR8n8, TR12n8, TR17n8, and TR18n8 that bear amino-acid substitutions, insertions, or deletions, may be prepared using synthetic double-stranded DNA fragments with sequences modified to encode such variations. Modified DNA sequences may be ordered from commercial DNA-synthesis providers; those skilled in the art can readily devise said sequence modifications, given the following caveats:

1. Modifications to the DNA fragment sequences should not remove existing recognition sequences for restriction enzymes Mlyl, NcoI-HF, Xhol, or SpeI-HF. Nor should the modifications introduce additional recognition sites for said enzymes.

2. Pairs of DNA subsequences present in the synthetic DNA fragments that are used to assemble DNA fragments must be kept identical to each other. For example, if the identical underlined subsequences as shown in TR8_1-2 and TR8_3-4 are to be modified, care must be taken to ensure that these two sequence regions remain identical after the modification. Likewise, the identical boldfaced subsequences shown in TR8_1-2 and TR8_3-4 must remain identical to each other in any proposed sequence modification. The analogous pairs of subsequences used for assembly of the pairs of fragments [TR12_1-2 and TR12_3-4], [TR18n8_1-2 and TR18n8_3-4], and [TR17n8_1-2 and TR17n8_3-4] and highlighted in the same way.

### Example 4: Recombinant Expression of Material-Forming Polypeptides

Example 4 provides methods of preparations of material-forming polypeptides as described herein. For example, when transformed with a plasmid that encodes a water-insoluble recombinant polypeptide, such as plasmid pET-14b-TR8n8, pET-14b-TR12n8, pET-14b-TR17n8, or pET-14b-TR18n8, laboratory strains of the bacterium E. coli can accumulate large amounts of the said polypeptide as intracellular inclusion bodies. The polypeptides as described herein may be isolated from the resulting cellular material using a variety of mechanical and solvent-based methods. Those skilled in the art will realize that a range of E. coli strains, media, and culture conditions can be used to achieve the production of intracellular recombinant polypeptides; an example is as the following but it is not intended to limit the scope of the disclosure. Given a sequence-verified, pET-14b-based expression vector for the desired polypeptide sequence, recombinant E. coli cells containing the polypeptide were prepared as follows:

1. A recombinant expression host was prepared with the following steps. A competent cell aliquot of E. coli strain BL21 (DE3) was transformed with the expression vector according to the instructions of the competent-cell supplier (e.g., EMD Millipore) and the transformation mixture was plated on an LB/agar plate supplemented with 100 μg/mL carbenicillin. The resulting plate was incubated at 34° C. for 18-22 hours until distinct colonies were visible. One colony was picked and transferred into a 4-mL LB media culture with 200 μg/mL of carbenicillin in a 14-mL disposable culture tube. The culture tube was incubated at 37° C. and 200 rpm for 12-16 hours until turbid. This culture was mixed with sterilized aqueous glycerol (50% v/v) at a 1:1 volume ratio in a cryotube and stored at −80° C.

2. A solid-format seed culture of the expression strain was grown with the following steps. The frozen cryostock made in step 1 was streaked onto an LB/agar plate supplemented with 100 μg/mL carbenicillin. The resulting plate was incubated at 34° C. for 18-22 hours until colonies were visible. All colonies were resuspended by adding 7 mL of fresh, sterile 4×LB medium (tryptone 40 g/L, yeast extract 20 g/L, NaCl 10 g/L) onto the plate, and then the colonies were gently scraped from the surface of the plate with a sterile spreading tool until the colonies were resuspended in the liquid phase. The liquid phase containing the resuspended colonies was decanted or pipetted out from the plate into a sterile tube. The optical density of the resulting cell slurry measured at 600 nm (OD600) was kept at the level of about 3.0-10 absorbance units, as extrapolated from measurements of samples that had been diluted such that their measured OD600 values were between 0.1-1.0 absorbance units.

3. 7 mL of seed slurry from step 2 was added to 150 mL of sterile 4×LB medium supplemented with 100 μg/mL carbenicillin in a 500-mL unbaffled Erlenmeyer flask. The resulting flask was incubated at 34° C. and 300 rpm for 24-30 hours. After this period of incubation, the dilution-extrapolated OD600 was about 2.5-3.5, and the pH was about 7.5-9.0. The cells were harvested by centrifugation at 5300 RPM (revolutions per minute) (6100 rcf, relative centrifugal force) for 20 minutes and decanting the supernatant. The resulting wet cell mass was about 2-3 g. The resulting cell pellets were frozen at −20° C. until purification.

### Example 5: Purification of Material-Forming Polypeptides

Example 5 provided methods for purifications of the polypeptides prepared according to the methods in Example 4. The purification method described herein is to extract polypeptide from dried cells using dimethyl sulfoxide (DMSO), remove cell debris by centrifugation or filtration, and then selectively precipitate the structural polypeptide using an antisolvent such as water, leaving much of the endogenous E. coli material in the DMSO-containing solution. Given a sample of E. coli cell paste containing a recombinant SRT polypeptide, the polypeptide was isolated as follows:

1. The polypeptides were extracted from the cell paste into DMSO with the following steps. To 2.5 g cell paste in 200-mL Erlenmeyer flask, was added 25 mL of DMSO and then the mixture was stirred for 30 minutes at room temperature. The resulting mixture was transferred to a 25-mL glass round-bottom flask and tip-sonicated (Branson 250, Tip 102C) for 1.5 minutes of total sonication time with a pulse mode (10 seconds on & 10 seconds off). The sonicated DMSO/cell mixture was poured back to a 200-mL Erlenmeyer flask and placed on a hot plate with magnetic stirring capabilities. The flask was covered with foil. With stirring, the temperature of the DMSO was brought to a stable 80° C. and continued stirring and heating for 30 minutes. Then the temperature was lowered to 30° C. and continued incubating for 20 minutes.

2. The warm DMSO mixture of Step 1 was transferred into a centrifuge tube and span at 5300 RPM (6100 rcf) in a centrifuge at 40° C. The supernatant was transferred to new tubes and centrifuged again using the same parameters. The supernatant showed transmission near 100% (absorbance or scattering near 0%) in a spectrometer at 600 nm. The DMSO supernatant was retained and the pellet was discarded.

3. The recombinant polypeptide was recovered with the following steps. The cleared DMSO supernatant from Step 2 was transferred into a 500-mL Erlenmeyer flask. 75 mL ultrapure water was added to the flask, and the resulting mixture was stirred overnight at room temperature. The recovery mixture (about 100-mL) was centrifuged at 10,000 RPM (17,700 rcf) for 30 minutes at 30° C. The supernatant was discarded and the pellet was retained.

4. The recovered polypeptide was then washed with the following steps. To the pellet collected in Step 3, was added 400 mL ultrapure water and the resulting mixture was incubated at least 12 hours at room temperature with stirring. The pellet was collected by centrifuging 10,000 RPM (17,700 rcf) for 30 minutes at 30° C. The supernatant was discarded. The 400-mL water wash as described herein was repeated and the pellet was collected again, using a 1-hour incubation. Finally, the pellet was resuspended in 50 mL ultrapure water and centrifuged again to collect the pellet in a 50-mL conical tube. The tube was open and inverted for 30 minutes to drain any remaining water. The tube was then recapped and frozen at −80° C. for at least 15 minutes.

5. Holes were made on the tube cap from Step 4 and the water-washed polypeptide material for 12-16 hours was lyophilized until completely dry. A Labconco FreeZone 6 plus or the like was used at this step at the conditions: vacuum 0.014 mBar, collector at −87° C.

FIG. 4 depicts the polypeptide purification as described herein. Dried cell paste containing the material-forming polypeptide was heated with DMSO to extract polypeptides into the solution. Residual cell debris were removed by centrifugation. The DMSO supernatant was mixed with water to precipitate the material-forming polypeptide. The isolated polypeptide was isolated by centrifugation. Finally, the isolated polypeptide was washed with water three times, and then dried prior to additional processing.

### Example 6: Preparation of Polypeptide Films for Transparency Testing

Example 6 provides methods of preparing polypeptide films as described herein for transparency testing. Films with a thickness of about 100 UM were prepared from these polypeptide materials by casting from solution as follows:

The polypeptide was dissolved with the following steps. 35 mg of lyophilized polypeptide material was weighed out and transferred into a microcentrifuge tube. To the microcentrifuge tube, was added 500 μL of 1,1,1,3,3,3-hexafluoroisopropanol (HFIP), and the tube was sealed with a lid and incubated at room temperature for 1 hour with occasional gentle inversion.

A film was cast with the following steps. Once the polypeptide was completely dissolved to form a solution from Step 1, 200-μL of the solution was pipetted into a PDMS (polydimethylsiloxane) mold (11.7 mm×12.2 mm×0.45 mm). The solvent was allowed to evaporate for 12-16 hours. Then, the film can be removed from the mold and subjected to transparency testing.

To hydrate a film produced in Step 1, the film was completely submerged in 10 mL of ultrapure water and incubated for at least 2 hours at room temperature. Then the hydrated film was moved into a fresh 1.5-mL volume of ultrapure water and incubated for 12-16 hours at room temperature before transparency measurements.

Following the steps described herein, polypeptide sequences TR12n8 (SEQ ID NO: 206), TR18n8 (SEQ ID NO:207), TR8n8 (SEQ ID NO:208), and TR17n8 (SEQ ID NO: 205) were formed into polypeptide films in both dry and hydrated forms.

### Example 7: Optical Transparency of Solvent-Cast Polypeptide Films

Example 7 provides methods of measuring the optical transparency of solvent-cast polypeptide films as described herein. Optical transparency of the polypeptide films may be measured, for example, using a Thermo Scientific Genesys 180 or the like in transmission mode and a wavelength range of 300-1100 nm using an interval of 2 nm. The films may be analyzed by affixing them to plastic cuvettes that had been modified by cutting holes in the plastic in the region of the spectrometer beam path using a Weller WLC100 soldering station. Testing of the empty modified cuvettes showed 100% transmission.

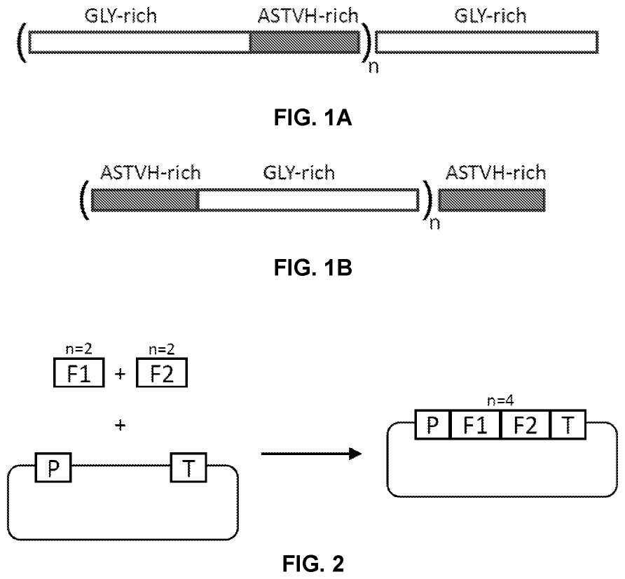

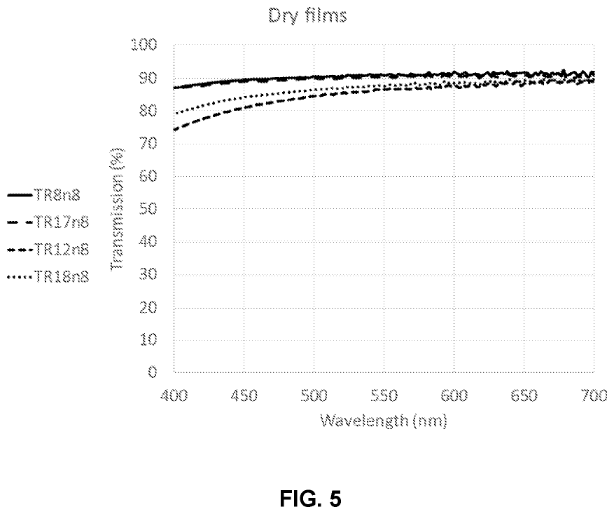

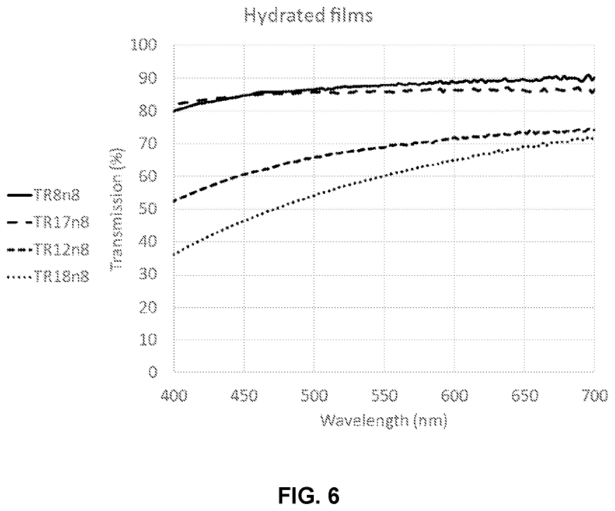

Both dry and hydrated forms of the polypeptide films prepared from the polypeptide sequences TR12n8, TR18n8, TR8n8, and TR17n8 as described here were tested for their optical transparency with the methods as described here. The unexpected and surprising results as shown in FIG. 5 and FIG. 6 demonstrated that films prepared from sequences TR8n8 and TR17n8, which were designed with the ASTVH-rich termini (FIG. 1B) offered improved optical transparency upon water hydration compared to films prepared sequences TR12n8 and TR18n8, which were produced with the GLY-rich termini (FIG. 1A). Films with a thickness of about 100 UM cast from HFIP solution and measured directly (FIG. 5, “Dry films”) show about 90% transmission across the visible spectrum for sequences with ASTVH-rich termini, while those with GLY-rich termini exhibit a reduction to about 75% transmission by 400 nm. When these same films are soaked in water and then blotted dry, the sequences with ASTVH-rich termini retain 80-90% transmission across the visible spectrum, while those with GLY-rich termini suffer from dramatically reduced transparency, down to 30-50% transmission around 400 nm (FIG. 6, “Hydrated films”). Sequences TR8n8 and TR18n8 use exactly the same ASTVH-rich and GLY-rich block sequences and differ only in their terminus architecture (ASTVH-rich termini or GLY-rich termini, respectively) and, therefore, the terminus architecture would likely be responsible for the large observed difference in optical transparency in the hydrated-state films, which was unexpected and surprising.

Various references and patents are disclosed herein, each of which are hereby incorporated by reference for the purpose that they are cited.

This description is not limited to the particular processes, compositions, polypeptides, or methodologies described, as these may vary. The terminology used in the description is for the purpose of describing the particular versions or embodiments only, and it is not intended to limit the scope of the embodiments described herein. Unless defined otherwise, all technical and scientific terms used herein have the same meanings as commonly understood by one of ordinary skill in the art. In some cases, terms with commonly understood meanings are defined herein for clarity and/or for ready reference, and the inclusion of such definitions herein should not necessarily be construed to represent a substantial difference over what is generally understood in the art. However, in case of conflict, the patent specification, including definitions, will prevail.

From the foregoing, it will be appreciated that various embodiments of the present disclosure have been described herein for purposes of illustration and that various modifications can be made without departing from the scope and spirit of the present disclosure. Accordingly, the various embodiments disclosed herein are not intended to be limiting.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A polypeptide having a formula:
A₁-(B₁-L₁-E₁-P₁)ₙ-B₁-G₁ Formula I,
wherein
A₁ is absent, is a methionine, or is an amino acid sequence 1 to 4 residues in length;
B₁ is an ASTVH-rich sequence amino acid sequence 6 to 17 residues in length comprising amino acids selected from the group consisting of alanine, serine, threonine, valine, histidine, glycine, glutamine, and proline, or any combination thereof.
L₁ is absent or is an amino acid sequence 1 to 7 residues in length comprising amino acids selected from the group consisting of proline, glycine, leucine, serine, and threonine, or any combination thereof;
E₁ is an GLY-rich amino acid sequence 8 to 58 residues in length comprising amino acids selected from the group consisting of glycine, leucine, tyrosine, phenylalanine, and proline, or any combination thereof;
P₁ is absent or is proline;
G₁ is absent or is an amino acid sequence 1 to 4 residues in length;
wherein n is 4 to 100; and
(i) wherein E₁ is YGFGGLYGGLFGGLGFG (SEQ ID NO:3) and B₁ comprises an amino acid sequence selected from the group consisting of SEQ ID NO: 11-88, or
(ii) wherein E₁ is YGYGGLFGGLFGGLGYG (SEQ ID NO:2) and B₁ comprises an amino acid sequence selected from the group consisting of SEQ ID NO: 12, 13, 17, 19-39, 41-52, 54-59, 61, 64-68, 70-78, 80, 82-84, and 88, or
(iii) wherein E₁ is YGYGGLYGGLYGGLGYG (SEQ ID NO:1) and B₁ comprises an amino acid sequence selected from the group consisting of SEQ ID NO: 12, 24, 27, 35, 37, 39, 55, 66, 67, 68, 71, 76, 82, and 83, or
(iv) wherein E₁ comprises an amino acid sequence selected from the group consisting of SEQ ID NO:90-204, B₁ comprises an amino acid sequence selected from the group consisting of SEQ ID NO: 13, 21-23, 25, 26, 29, 30, 32, 34, 36, 37, 39, 44-46, 48, 50-52, 55, 57, 58, 61, 64-68, 71, 72, 74, 76-78, 80-83, and 89, and L₁ is absent or is Pro.

2. The polypeptide of claim 1, wherein the polypeptide is a synthetic or recombinant supramolecular polypeptide.

3. The polypeptide of claim 1, wherein the A₁ is methionine (M).

4. The polypeptide of claim 1, wherein L₁ is selected from the group consisting of SEQ ID NOs: 4 to 10.

5. The polypeptide of claim 1, wherein G₁ is Thr-Ser (TS) or Pro-Thr-Ser (PTS).

6. The polypeptide of claim 1, wherein n is 4-20.

7. The polypeptide of claim 1, wherein A₁ is methionine (M), L₁ is SEQ ID NO:4, and G₁ is Pro-Thr-Ser (PTS).

8. The polypeptide of claim 1, wherein E₁ is YGYGGLFGGLFGGLGYG (SEQ ID NO:2) and B₁ comprises is SEQ ID NO:23.

9. The polypeptide of claim 8 comprising the amino acid sequence SEQ ID NO: 205.

10. A composition comprising a polypeptide of claim 1 in a solvent.

11. The composition of claim 10, wherein the polypeptide is formulated as an adhesive or film.

12. The composition of claim 10, wherein the polypeptide is formulated as a fiber.

13. The composition of claim 10, wherein the solvent is dimethyl sulfoxide, formic acid, 1,1,1,3,3,3-hexafluoro-2-propanol, aqueous ammonia, aqueous alkali-metal hydroxide, or aqueous urea.

14. The composition of claim 10, wherein the solvent is an ionic liquid.

15. The composition of claim 14, wherein the solvent is 1-ethyl-3-methylimidazolium acetate.
